(19) United States
(12) Patent Application Publication
(10) Pub. No.: US 2020/0369594 A1
(43) Pub. Date: Nov. 26, 2020

(54) COMPOUNDS COMPRISING SHORT-CHAIN FATTY ACID MOIETIES AND COMPOSITIONS AND METHODS THEREOF

(71) Applicant: CARNOT3, LLC, Boston, MA (US)

(72) Inventor: Andrew D. Levin, Newton, MA (US)

(21) Appl. No.: 16/967,778

(22) PCT Filed: Feb. 13, 2019
(86) PCT No.: PCT/US2019/017815

§ 371 Date: Aug. 6, 2020

Related U.S. Application Data
(60) Provisional application No. 62/710,478, filed on Feb. 16, 2018.

(51) Int. Cl. C07C 69/40

(52) U.S. Cl. CPC C07C 69/40

(57) ABSTRACT

Among other things, the present disclosure provides compounds and compositions that can effectively deliver short-chain fatty acid entities. In some embodiments, provided compounds and compositions are of improved properties and can provide improved administration and/or dosing regimens. In some embodiments, provided technologies are particularly useful for treating an inflammatory bowel disease. In some embodiments, provided technologies are particularly useful for treating a irritable bowel syndrome.

### REFERENCE TO RELATED APPLCIATION

This application claims the benefit of, and priority to, U.S. Provisional Patent Application No. 62/710,478, filed Feb. 16, 2018, the contents of which are incorporated herein by reference.

### BACKGROUND

Short-chain fatty acids are produced in human bodies and are involved in many biological pathways.

### SUMMARY

The present disclosure relates to compositions and/or compounds that deliver and/or comprise short-chain fatty acid entities. In some embodiments, provided technologies achieve delivery when administered orally. In some embodiments, provided technologies are particularly useful in the treatment of diseases, disorders, or conditions that involve inflammation (and/or cell proliferation). In particular embodiments, provided technologies are particularly useful in treatment of inflammatory conditions of the gastrointestinal tract such as, for example, inflammatory bowel diseases, irritable bowel syndrome, etc.

Among other things, the present disclosure provides technologies that address certain long-felt needs, including in particular as relate to administration of short-chain fatty acid entities to humans. For example, the present disclosure appreciates that observations of potentially beneficial impact of short-chain fatty acids on colonic health have been reported for many years (see, for example, van der Beek, et al., Role of short-chain fatty acids in colonic inflammation, carcinogenesis, and mucosal protection and healing, Nutrition Reviews Vol. 75(4):286-305). However, efforts to achieve clinical benefit through administration of such agents (whether by application or modulation of microbial populations that produce them or by administration, typically by enema, of compositions containing them) have generally not met with success. Indeed, a current review article reports that “[m]ost [randomized, double-blind, placebo-controlled] studies [in ulcerative colitis] report no significant different between [small chain fatty acid] application and placebo” (van der Beek, Nutrition Reviews Vol. 75(4):286-305, pg 293, citing Hamer H M, Jonkers D M, Vanhoutvin S A, et al. Effect of butyrate enemas on inflammation and antioxidant status in the colonic mucosa of patients with ulcerative colitis in remission. Clin Nutr. 2010; 29:738-744; Scheppach W. Treatment of distal ulcerative colitis with short-chain fatty acid enemas. A placebo-controlled trial. German-Austrian SCFA Study Group. Dig Dis Sci. 1996; 41:2254-2259; and Scheppach W, Muller J G, Boxberger F, et al. Histological changes in the colonic mucosa following irrigation with short-chain fatty acids. Eur J Gastroenterol Hepatol. 1997; 9:163-168.).

In some aspects, the present disclosure identifies the source(s) of certain problems encountered in prior efforts to effectively administer short-chain fatty acid entities, particularly to humans. In some embodiments, for example, the present disclosure appreciates that prior technologies may not have been able to administer such entities at high enough quantities to be efficacious. In some embodiments, the present disclosure appreciates that certain compositions, for example, those comprising free hydroxyl groups (e.g., of diols or polyols) and/or free carboxylic acid groups can have so high viscosity that they cannot be readily formulated and/or administered. Additionally or alternatively, in some embodiments, the present disclosure observes that certain compositions, for example, those having free carboxylic acid groups, can be very unpalatable, rendering oral administration difficult if not impossible. The present disclosure recognizes that prior technologies have typically administered short-chain fatty acids in relatively low dosages, via limited administration methods, and/or in particular forms or formats; for example, short chain fatty acids were often administered as free acids (or salt) via enema.

In some embodiments, the present disclosure encompasses the recognition that particular features of prior technologies may have resulted in ineffective delivery and/or otherwise have contributed to observed low (or absent) of efficacy of administered short-chain fatty acid entities, in particular for example, in the treatment of inflammatory conditions of the gastrointestinal tract, such as, for example, inflammatory bowel diseases, irritable bowel syndrome, etc.

In some embodiments, the present disclosure provides technologies that can effectively deliver short-chain fatty acid entities. In some embodiments, the present disclosure provides technologies that permit and/or achieve relatively high dosing of short-chain fatty acid entities to humans. In some embodiments, the present disclosure provides new and/or more effective therapeutics for certain conditions, disorders and/or diseases, e.g., inflammatory conditions of the gastrointestinal tract, such as inflammatory bowel diseases, irritable bowel syndrome, etc. In some embodiments, provided technologies achieve reduction in inflammation without general immune suppression. In some embodiments, provided compounds are metabolized to natural products.

In some embodiments, the present disclosure provides compounds of designed molecular structures, and compositions and methods thereof, for, e.g., effective delivery of short-chain fatty acids useful for treating a variety of conditions, disorders and/or diseases, e.g., inflammatory bowel diseases, irritable bowel syndrome, etc.

In some embodiments, the present disclosure provides compounds and compositions that are of significantly improved properties, e.g., taste, melting point, viscosity, etc., so that provided compounds and compositions can be readily formulated for efficient administration to a subject via a variety of methods including oral administration. In some embodiments, the present disclosure provides compounds and/or compositions that have greatly improved flow property and/or taste (e.g., no or tolerable bitterness) for formulation and/or oral administration. In some embodiments, provided compounds are suitable for direct oral administration. In some embodiments, provided compounds are of such flow property and/or taste so that they are suitable for direct oral administration by direct drinking by a subject. In some embodiments, provided compounds and compositions can be readily administered at significantly higher unit doses. In some embodiments, provided compounds can be administered in high quantities, e.g., at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4 or 5 g/kg.

In some embodiments, the present disclosure provides a compound having the structure of formula I:

R^{A}-L-R^{B}, I

or a salt thereof, wherein:
R^{A} is

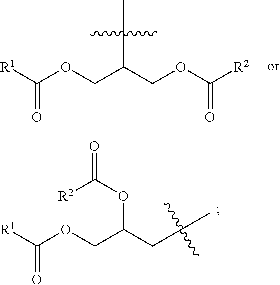

R^{B} is

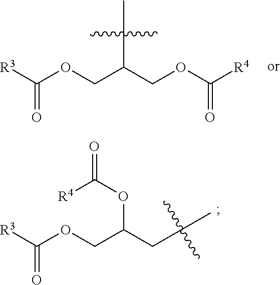

each of R¹, R², R³ and R⁴ is independently C₁₋₃ alkyl;
L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with bivalent C₁₋₄ aliphatic, —O—, —C(O)—, C(O)O—, —S—, —N(R′)—, or —C(O)N(R′)—;
R′ is R or —C(O)R;
each R is independently hydrogen, or an optionally substituted group selected from C₁₋₆ aliphatic, C₁₋₆ heteroaliphatic having 1-5 hetereoatoms independently selected from nitrogen, oxygen, and sulfur, phenyl, 5-6 membered heteroaryl having 1-5 hetereoatoms independently selected from nitrogen, oxygen, and sulfur, and 3-6 membered heterocyclyl having 1-5 hetereoatoms independently selected from nitrogen, oxygen, and sulfur.

In some embodiments, L is an optionally substituted linear or branched, bivalent C₁₋₆ aliphatic, wherein one or more methylene units of the aliphatic and heteroaliphatic are optionally and independently replaced with bivalent C₁₋₄ aliphatic, —O—, —C(O)—, —C(O)O—, —S—, —N(R′)—, or —C(O)N(R′)—. In some embodiments, L is linear or branched, bivalent C₁₋₆ aliphatic, wherein one or more methylene units of the aliphatic and heteroaliphatic are optionally and independently replaced with bivalent C₁₋₄ aliphatic, —O—, —C(O)—, —C(O)O—, —S—, —N(R′)—, or —C(O)N(R′)—. In some embodiments, at least one methylene unit is replaced by —O—. In some embodiments, at least one methylene unit is replaced by —C(O)—. In some embodiments, at least one methylene unit is replaced by —C(O)O—. In some embodiments, at least one methylene unit is replaced by —N(R′)—. In some embodiments, at least one methylene unit is replaced by —N(R)—. In some embodiments, at least one methylene unit is replaced by —NH—. In some embodiments, at least one methylene unit is replaced by —C(O)N(R′)—. In some embodiments, at least one methylene unit is replaced by —C(O)N(R)—. In some embodiments, at least one methylene unit is replaced by —C(O)NH—. In some embodiments, at least two methylene units are independently replaced by —O—, —C(O)—, —C(O)O—, —S—, —N(R′)—, or —C(O)N(R′)—. In some embodiments, at least two methylene units are independently replaced by —C(O)O—. In some embodiments, L is optionally substituted alkylene dicarboxyl. In some embodiments, each of the two terminal methylene group of L is independently replaced by —O—, —C(O)—, —C(O)O—, —S—, —N(R′)—, or C(O)N(R′)—. In some embodiments, each of the two terminal methylene group of L is independently replaced by —O—. In some embodiments, each of the two terminal methylene group of L is independently replaced by —C(O)O—. In some embodiments, each of the two terminal methylene group of L is independently replaced by —N(R′)—. In some embodiments, each of the two terminal methylene group of L is independently replaced by —NH—. In some embodiments, each of the two terminal methylene group of L is independently replaced by —C(O)N(R′)—. In some embodiments, each of the two terminal methylene group of L is independently replaced by —C(O)N(R)—. In some embodiments, each of the two terminal methylene group of L is independently replaced by —C(O)NH—. In some embodiments, L is a dicarboxyl group from a TCA cycle di-acid or tri-acid. In some embodiments, L is a dicarboxyl group of a TCA cycle di-acid, e.g., —OC(O)—CH₂CH₂C(O)O—. In some embodiments, L is —OC(O)—CH₂CH₂C(O)O—.

In some embodiments, a provided compound has the structure of formula I-c or a pharmaceutically acceptable salt thereof:

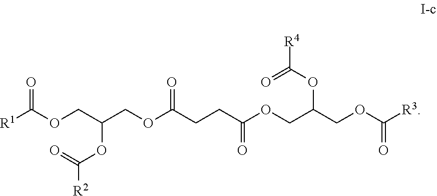

In some embodiments, a provided compound has the structure of formula I-d or a pharmaceutically acceptable salt thereof:

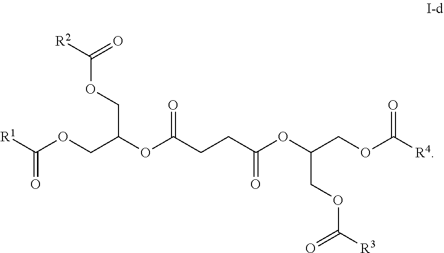

In some embodiments, a provided compound has the structure of formula I-e or a pharmaceutically acceptable salt thereof:

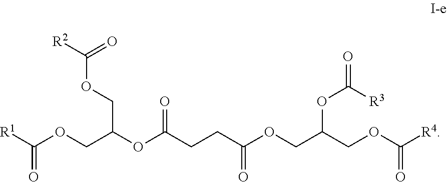

In some embodiments, R¹, R², R³ and R⁴ are the same. In some embodiments, at least one of R¹, R²,R³ and R⁴ is different from at least another R¹, R², R³ and R⁴. In some embodiments, two of R1, ^{R2}, and R⁴ are the same. In some embodiments, three of R¹, R², R³ R³ and R⁴ are the same. In some embodiments, all of R¹, R², R³ and R⁴ are the same.

In some embodiments, one of R¹, R², R³ and R⁴ is methyl. In some embodiments, one of R¹, R², R³ and R⁴ is ethyl. In some embodiments, one of R¹, R², R³ and R⁴ is propyl. In some embodiments, one or R¹, R², R³ and R⁴ is n-propyl.

In some embodiments, each of R¹, R², R³ and R⁴ is n-propyl.

In some embodiments, a provided compound is compound I-I:

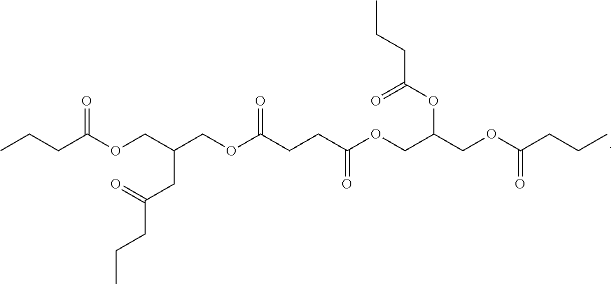 [SMILES: CCCC(=O)CC(COC(=O)CCC)COC(=O)CCC(=O)OCC(COC(=O)CCC)OC(=O)CCC]

In some embodiments, a provided compound is compound 1-2:

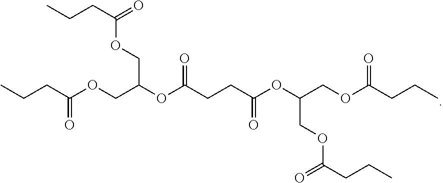 [SMILES: CCCC(=O)OCC(COC(=O)CCC)OC(=O)CCC(=O)OC(COC(=O)CCC)COC(=O)CCC]

In some embodiments, the compounds include one or more atoms that are enriched for an isotope. For example, the compounds may have one or more hydrogen atoms replaced with deuterium or tritium.

In some embodiments, the present disclosure provides compositions comprising provided compounds. In some embodiments, a provided composition is a pharmaceutical composition, comprising a provided compound or a pharmaceutically salt thereof, and optionally a pharmaceutically acceptable carrier. In some embodiments, a composition consists of a provided compound. In some embodiments, a composition consisting of a provided compound is of sufficiently low viscosity and acceptable taste so that it can be directly administered by drinking.

In some embodiments, the present disclosure provides methods for increasing level of a short-chain fatty acid in a system, comprising administering to the system a provided compound. In some embodiments, the present disclosure provides methods for delivering a short-chain fatty acid to a system, comprising administering to the system a provided compound. In some embodiments, a system is a cell, tissue, organ, or subject. In some embodiments, the present disclosure provides methods for increasing level of a short-chain fatty acid in a cell, tissue, and/or organ, comprising contacting a cell, tissue, and/or organ with a provided compound. In some embodiments, the present disclosure provides methods for increasing level of a short-chain fatty acid in a cell, tissue, and/or organ of a subject, comprising administering to the subject a provided compound or composition. Without the intention to be limited by any theory, in some embodiments, after administration to a system, a provided compound is hydrolyzed and/or otherwise metabolized to provide one or more short-chain fatty acids.

In some embodiments, the present disclosure provides methods for preventing and/or treating a number of conditions, disorders or diseases that are associated with abnormal levels of one or more short-chain fatty acids in a subject, and/or that benefit from increased levels of short-chain fatty acids, comprising administering to a subject susceptible thereto or suffering therefrom a provided compound or composition.

In some embodiments, a condition, disorder or disease is an inflammatory bowel disease. In some embodiments, a condition, disorder or disease is ulcerative colitis. In some embodiments, a condition, disorder or disease is diversion colitis. In some embodiments, a condition, disorder or disease is radiation proctitis. In some embodiments, a condition, disorder or disease is radiation colitis. In some embodiments, a condition, disorder or disease is pouchitis. In some embodiments, a condition, disorder or disease is Crohn's disease. In some embodiments, a condition, disorder or disease is collagenous colitis. In some embodiments, a condition, disorder or disease is lymphocytic colitis.

In some embodiments, a condition, disorder or disease is an irritable bowel syndrome. In some embodiments, an irritable bowel syndrome is diarrhea type (IBS-D). In some embodiments, an irritable bowel syndrome is constipation type (IBS-C). In some embodiments, an irritable bowel syndrome is mixed type (IBS-M), wherein both diarrhea and constipation are common. In some embodiments, an irritable bowel syndrome is IBS-U, wherein neither diarrhea nor constipation is common.

Among other things, provided compounds and compositions provide greatly improved flexibility with respect to administration methods and/or dosage regimens, compared to previously known compounds and compositions, such as acids and/or salts of short-chain fatty acids. In some embodiments, provided compounds and compositions are administered through non-enema pathways. In some embodiments, provided compounds and compositions are administered orally. In some embodiments, provided compounds and compositions are administered by direct drinking. In some embodiments, provided compounds and compositions are administered by enema.

In some embodiments, provided technologies provide dosage regimen that comprise high unit doses and/or total doses, e.g., within a day, week, and/or month. In some embodiments, provided compounds can be administered at at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, or 5 g/kg per day, either as a single dose or as multiple doses. In some embodiments, a unit dose is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, or 5 g. In some embodiments, a single dose is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, or 5 g. In some embodiments, a daily total dose is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, or 5 g. In some embodiments, a dosage regimen comprises daily administration of at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, or 5 g/kg per day of a provided compound for at least about 1, 2, 3, 4, 5, 6, 7, 10, 14, 15, 20, 21, 25, 28, 30, 31, 35, 40, 42, 45, 49, 50, 60, 70, 80, 90, 100, 150, 200, 300, 350, 365, or 400 days.

### BRIEF DESCRIPTION OF THE DRAWINGS

FIG. 1 is graph of NMR data of compound I-1.

FIG. 2 is graph of colon length in mice with DSS-induced colitis after administration of compounds of the invention. Provided compounds, as illustrated by Compound I-1, can significantly restore colon length in dextran sulfate sodium (DSS)-induced colitis in mice. Naive: no treatment. For vehicle, CsA, and Compound I-1, animals were treated with DSS and vehicle (water), CsA, and Compound I-1, respectively.

FIG. 3 is graph of body weight in mice with DSS-induced colitis after administration of compounds of the invention.

FIG. 4 is a graph of change in body weight over time in animals with TNBS-induced colitis after administration of CV-8784 or control compounds.

FIG. 5 is a graph of colon weight per length in animals with TNBS-induced colitis after administration of CV-8784 or control compounds.

FIG. 6 is a graph of the overall inflammation score in colon tissue in animals with TNBS-induced colitis after administration of CV-8784 or control compounds.

FIG. 7 is a graph of gland loss in colon tissue in animals with TNBS-induced colitis after administration of CV-8784 or control compounds.

FIG. 8 is a graph of tissue erosion in colon tissue in animals with TNBS-induced colitis after administration of CV-8784 or control compounds.

FIG. 9 is a graph of edema in colon tissue in animals with TNBS-induced colitis after administration of CV-8784 or control compounds.

FIG. 10 is a graph of neutrophil infiltration in colon tissue in animals with TNBS-induced colitis after administration of CV-8784 or control compounds.

FIG. 11 is a graph of lymhocyte aggregation in colon tissue in animals with TNBS-induced colitis after administration of CV-8784 or control compounds.

FIG. 12 is a graph of change in body weight over time in animals with DSS-induced colitis after administration of CTP-06 or control compounds.

FIG. 13 is a graph of colon length in animals with DSS-induced colitis after administration of CTP-06 or control compounds.

FIG. 14 is a graph of colon content in animals with DSS-induced colitis after administration of CTP-06 or control compounds.

FIG. 15 is a graph of intestinal transit of a glass bead in animals after administration of CV-8784 or control compounds.

FIG. 16 is a graph of intestinal transit of a charcoal solution in an animals after administration of CV-8784 or control compounds.

### DETAILED DESCRIPTION OF CERTAIN EMBODIMENTS

### 1. Definitions

As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

Aliphatic: As used herein, “aliphatic” means a straight-chain (i.e., unbranched) or branched, substituted or unsubstituted hydrocarbon chain that is completely saturated or that contains one or more units of unsaturation, or a monocyclic hydrocarbon, bicyclic hydrocarbon, or polycyclic hydrocarbon that is completely saturated or that contains one or more units of unsaturation that has a single point of attachment to the rest of the molecule. Unless otherwise specified, aliphatic groups contain 1-100 aliphatic carbon atoms. In some embodiments, aliphatic groups contain 1-20 aliphatic carbon atoms. In other embodiments, aliphatic groups contain 1-10 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof.

Alkyl: As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain), and alternatively, about 1-10. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

Alkenyl: As used herein, the term “alkenyl” refers to an alkyl group, as defined herein, having one or more double bonds.

Alkynyl: As used herein, the term “alkynyl” refers to an alkyl group, as defined herein, having one or more triple bonds.

Aryl: The term “aryl”, as used herein, used alone or as part of a larger moiety as in “aralkyl,” “aralkoxy,” or “aryloxyalkyl,” refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like, which may bear one or more substituents. Also included within the scope of the term “aryl,” as it is used herein, is a group in which an aromatic ring is fused to one or more nonaromatic rings, such as indanyl, phthalimidyl, naphthimidyl, phenanthridinyl, or tetrahydronaphthyl, and the like.

Cycloaliphatic: The term “cycloaliphatic,” “carbocycle,” “carbocyclyl,” “carbocyclic radical,” and “carbocyclic ring,” are used interchangeably, and as used herein, refer to saturated or partially unsaturated, but non-aromatic, cyclic aliphatic monocyclic, bicyclic, or polycyclic ring systems, as described herein, having, unless otherwise specified, from 3 to 30 ring members. Cycloaliphatic groups include, without limitation, cyclopropyl, cyclobutyl, cyclopentyl, cyclopentenyl, cyclohexyl, cyclohexenyl, cycloheptyl, cycloheptenyl, cyclooctyl, cyclooctenyl, norbornyl, adamantyl, and cyclooctadienyl. In some embodiments, a cycloaliphatic group has 3-6 carbons. In some embodiments, a cycloaliphatic group is saturated and is cycloalkyl. The term “cycloaliphatic” may also include aliphatic rings that are fused to one or more aromatic or nonaromatic rings, such as decahydronaphthyl or tetrahydronaphthyl. In some embodiments, a cycloaliphatic group is bicyclic. In some embodiments, a cycloaliphatic group is tricyclic. In some embodiments, a cycloaliphatic group is polycyclic. In some embodiments, “cycloaliphatic” refers to C₃-C₆ monocyclic hydrocarbon, or C₈-Cᵢₒ bicyclic or polycyclic hydrocarbon, that is completely saturated or that contains one or more units of unsaturation, but which is not aromatic, that has a single point of attachment to the rest of the molecule, or a C₉-C₁₆ polycyclic hydrocarbon that is completely saturated or that contains one or more units of unsaturation, but which is not aromatic, that has a single point of attachment to the rest of the molecule.

Heteroaliphatic: The term “heteroaliphatic”, as used herein, is given its ordinary meaning in the art and refers to aliphatic groups as described herein in which one or more carbon atoms are independently replaced with one or more heteroatoms (e.g., oxygen, nitrogen, sulfur, silicon, phosphorus, and the like). In some embodiments, one or more units selected from C, CH, CH₂, and CH₃ are independently replaced by one or more heteroatoms (including oxidized and/or substituted form thereof). In some embodiments, a heteroaliphatic group is heteroalkyl. In some embodiments, a heteroaliphatic group is heteroalkenyl.

Heteroalkyl: The term “heteroalkyl”, as used herein, is given its ordinary meaning in the art and refers to alkyl groups as described herein in which one or more carbon atoms are independently replaced with one or more heteroatoms (e.g., oxygen, nitrogen, sulfur, silicon, phosphorus, and the like). Examples of heteroalkyl groups include, but are not limited to, alkoxy, poly(ethylene glycol)-, alkyl-substituted amino, tetrahydrofuranyl, piperidinyl, morpholinyl, etc.

Heteroaryl: The terms “heteroaryl” and “heteroar”, as used herein, used alone or as part of a larger moiety, e.g., “heteroaralkyl,” or “heteroaralkoxy,” refer to monocyclic, bicyclic or polycyclic ring systems having a total of five to thirty ring members, wherein at least one ring in the system is aromatic and at least one aromatic ring atom is a heteroatom. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar”, as used herein, also include groups in which a heteroaromatic ring is fused to one or more aryl, cycloaliphatic, or heterocyclyl rings, where the radical or point of attachment is on the heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3b]-1,4oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

Heteroatom: The term “heteroatom”, as used herein, means an atom that is not carbon or hydrogen. In some embodiments, a heteroatom is boron, oxygen, sulfur, nitrogen, phosphorus, or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.). In some embodiments, a heteroatom is selected from oxygen, nitrogen and sulfur.

Heterocycle: As used herein, the terms “heterocycle,” “heterocyclyl,” “heterocyclic radical,” and “heterocyclic ring”, as used herein, are used interchangeably and refer to a monocyclic, bicyclic or polycyclic ring moiety (e.g., 3-30 membered) that is saturated or partially unsaturated and has one or more heteroatom ring atoms. In some embodiments, a heterocyclyl group is a stable 5to 7membered monocyclic or 7to 10membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur and nitrogen, the nitrogen may be N (as in 3,4dihydro-2Hpyrrolyl), NH (as in pyrrolidinyl), or ⁺NR (as in Nsubstituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3Hindolyl, chromanyl, phenanthridinyl, or tetrahydroquinolinyl. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Partially unsaturated: As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

Protecting Group: The phrase “protecting group,” as used herein, refers to temporary substituents which protect a potentially reactive functional group from undesired chemical transformations. Examples of such protecting groups include esters of carboxylic acids, silyl ethers of alcohols, and acetals and ketals of aldehydes and ketones, respectively. A “Si protecting group” is a protecting group comprising a Si atom, such as Si-trialkyl (e.g., trimethylsilyl, tributylsilyl, t-butyldimethylsilyl), Si-triaryl, Si-alkyl-diphenyl (e.g., t-butyldiphenylsilyl), or Si-aryl-dialkyl (e.g., Si-phenyldialkyl). Generally, a Si protecting group is attached to an oxygen atom. The field of protecting group chemistry has been reviewed (Greene, T. W.; Wuts, P. G. M. Protective Groups in Organic Synthesis, 2nd ed.; Wiley: New York, 1991). Such protecting groups (and associated protected moieties) are described in detail below.

Protected hydroxyl groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitably protected hydroxyl groups further include, but are not limited to, esters, carbonates, sulfonates, allyl ethers, ethers, silyl ethers, alkyl ethers, arylalkyl ethers, and alkoxyalkyl ethers. Examples of suitable esters include formates, acetates, proprionates, pentanoates, crotonates, and benzoates. Specific examples of suitable esters include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benzylbenzoate, 2,4,6-trimethylbenzoate. Examples of suitable carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of suitable silyl ethers include trimethylsilyl, triethylsilyl, t-butyldimethylsilyl, t-butyldiphenylsilyl, triisopropylsilyl ether, and other trialkylsilyl ethers. Examples of suitable alkyl ethers include methyl, benzyl, p-methoxybenzyl, 3,4-dimethoxybenzyl, trityl, t-butyl, and allyl ether, or derivatives thereof. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyran-2-yl ether. Examples of suitable arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, 2- and 4-picolyl ethers.

Protected amines are well known in the art and include those described in detail in Greene (1999). Suitable mono-protected amines further include, but are not limited to, aralkylamines, carbamates, allyl amines, amides, and the like. Examples of suitable mono-protected amino moieties include t-butyloxycarbonylamino (—NHBOC), ethyloxycarbonylamino, methyloxycarbonylamino, trichloroethyloxycarbonylamino, allyloxycarbonylamino (—NHAlloc), benzyloxocarbonylamino (—NHCBZ), allylamino, benzylamino (—NHBn), fluorenylmethylcarbonyl (—NHFmoc), formamido, acetamido, chloroacetamido, dichloroacetamido, trichloroacetamido, phenylacetamido, trifluoroacetamido, benzamido, t-butyldiphenylsilyl, and the like. Suitable di-protected amines include amines that are substituted with two substituents independently selected from those described above as mono-protected amines, and further include cyclic imides, such as phthalimide, maleimide, succinimide, and the like. Suitable di-protected amines also include pyrroles and the like, 2,2,5,5-tetramethyl-[1,2,5]azadisilolidine and the like, and azide.

Protected aldehydes are well known in the art and include those described in detail in Greene (1999). Suitable protected aldehydes further include, but are not limited to, acyclic acetals, cyclic acetals, hydrazones, imines, and the like. Examples of such groups include dimethyl acetal, diethyl acetal, diisopropyl acetal, dibenzyl acetal, bis(2-nitrobenzyl) acetal, 1,3-dioxanes, 1,3-dioxolanes, semicarbazones, and derivatives thereof.

Protected carboxylic acids are well known in the art and include those described in detail in Greene (1999). Suitable protected carboxylic acids further include, but are not limited to, optionally substituted C₁₋₆ aliphatic esters, optionally substituted aryl esters, silyl esters, activated esters, amides, hydrazides, and the like. Examples of such ester groups include methyl, ethyl, propyl, isopropyl, butyl, isobutyl, benzyl, and phenyl ester, wherein each group is optionally substituted. Additional suitable protected carboxylic acids include oxazolines and ortho esters.

Protected thiols are well known in the art and include those described in detail in Greene (1999). Suitable protected thiols further include, but are not limited to, disulfides, thioethers, silyl thioethers, thioesters, thiocarbonates, and thiocarbamates, and the like. Examples of such groups include, but are not limited to, alkyl thioethers, benzyl and substituted benzyl thioethers, triphenylmethyl thioethers, and trichloroethoxycarbonyl thioester, to name but a few.

Substitution or Optionally Substituted: As described herein, compounds of the disclosure may contain optionally substituted and/or substituted moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents include halogen; —(CH₂)₀₋₄R°; —(CH₂)₀₋₄OR°; —O(CH₂)₀₋₄R°), —O—(CH₂)₀₋₄C(O)OR°; —(CH₂)₀₋₄CH(OR°₂; —(CH₂)₀₋₄Ph, which may be substituted with R°; —(CH₂)₀₋₄O(CH₂)₀₋₁Ph which may be substituted with R°; —CH═CHPh, which may be substituted with R°; —(CH₂)₀₋₄O(CH₂)₀₋₁-pyridyl which may be substituted with R°; —NO₂; —CN; —N₃; —(CH₂)₀₋₄N(R°₂; —(CH₂)₀₋₄N(R°)C(O)R°; —N(R°)C(S)R°; —(CH₂)₀₋₄N(R°)C(O)NR°₂; —N(R°)C(S)NR°₂; —(CH₂)₀₋₄N(R°; —C(O)OR°; —N(R°)N(R°)C(O)R°; —N(R°)N(R°)C(O)NR°₂; —N(R°)N(R°)C(O)OR°; —(CH₂)₀₋₄C(O)R°; —C(S)R°; —(CH₂)₀₋₄C(O)OR°; —(CH₂)₀₋₄C(O)SR°; —(CH₂)₀₋₄C(O)OSiR°₃; —(CH₂)₀₋₄OC(O)R°; —OC(O)(CH₂)₀₋₄SR, SC(S)SR°; —(CH₂)₀₋₄SC(O)R°; —(CH₂)₀₋₄C(O)NR°₂; —C(S)NR°₂; —C(S)SR°; —SC(S)SR°, —(CH₂)₀₋₄OC(O)NR°₂; —C(O)N(OR°)R°; —C(O)C(O)R°; —C(O)CH₂C(O)R°; —C(NOR°)R°; —(CH₂)₀₋₄SSR°; —(CH₂)₀₋₄S(O)₂R°; —(CH₂)₀₋₄S(O)₂OR°; —(CH₂)₀₋₄OS(O)₂R°; —S(O)₂NR°₂; —(CH₂)₀₋₄S(O)R°; —N(R°)S(O)₂NR°₂; —N(R°)S(O)₂R°; —N(OR°)R°; —C(NH)NR°₂; —P(O)₂R°; —P(O)R°₂; —OP(O)R°₂; —OP(O)(OR°₂; SiR°₃; OSiR°₃; —(C₁₋₄ straight or branched)alkylene)O—N(R°)₂; or —(C₁₋₄ straight or branched)alkylene)C(O)O—N(R°₂, wherein each R° may be substituted as defined below and is independently hydrogen, C₁₋₆ aliphatic, —CH₂Ph, —O(CH₂)₀₋₁Ph, —CH₂-(5-6 membered heteroaryl ring), or a 5-6membered saturated, partially unsaturated, or aryl ring having 0-4 heteroatoms independently selected from nitrogen, oxygen, or sulfur, or, notwithstanding the definition above, two independent occurrences of R°, taken together with their intervening atom(s), form a 3-12membered saturated, partially unsaturated, or aryl mono or bicyclic ring having 0-4 heteroatoms independently selected from nitrogen, oxygen, or sulfur, which may be substituted as defined below.

Suitable monovalent substituents on R° (or the ring formed by taking two independent occurrences of R° together with their intervening atoms), are independently halogen, (CH₂)₀₋₂R●, (halonR●), —(CH₂)₀₋₂OH, —(CH₂)₀₋₂OR●, —(CH₂)₀₋₂CH(OR●)₂; —O(haloR●), —CN, —N₃, —(CH₂)₀₋₂C(O)R●, —(CH₂)₀₋₂C(O)OH, —(CH₂)₀₋₂C(O)OR●, —(CH₂)₀₋₂SR●, (CH₂)₀₋₂SH, —(CH₂)₀₋₂NH₂, —(CH₂)₀₋₂NHR●, —(CH₂)₀₋₂NR●₂, —NO₂, —SiR●₃, — OSiR●₃, —C(O)SR●, —(C₁₋₄ straight or branched alkylene)C(O)OR●, or —SSR● wherein each R● is unsubstituted or where preceded by “halo” is substituted only with one or more halogens, and is independently selected from C₁₋₄ aliphatic, CH₂Ph, —O—(CH₂)₀₋₁Ph, or a 5-6membered saturated, partially unsaturated, or aryl ring having 0-4 heteroatoms independently selected from nitrogen, oxygen, or sulfur. Suitable divalent substituents on a saturated carbon atom of R° include ═O and ═S.

Suitable divalent substituents include the following: ═O—, ═S, ═NNR*₂, ═NNHC(O)R*, ═NNHC(O)OR*, ═NNHS(O)₂R*, ═NR*, ═NOR*, 13 O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃—O—, wherein each independent occurrence of R* is selected from hydrogen, C₁₋₆ aliphatic which may be substituted as defined below, or an unsubstituted 5-6membered saturated, partially unsaturated, or aryl ring having 0-4 heteroatoms independently selected from nitrogen, oxygen, or sulfur.

Suitable substituents on the aliphatic group of R* include halogen, —R●, -(haloR●), OH, —OR●, —O—(halol●), —CN, —C(O)OH, —C(O)OR●, —NH₂, NHR●,—NR●₂, or —NO₂, wherein each r is unsubstituted or where preceded by “halo” is substituted only with one or more halogens, and is independently C₁₋₄ aliphatic, —CH₂Ph, —O—(CH₂)₀₋₁Ph, or a 5-6membered saturated, partially unsaturated, or aryl ring having 0-4 heteroatoms independently selected from nitrogen, oxygen, or sulfur.

In some embodiments, suitable substituents on a substitutable nitrogen include —R†, —NR†₂, —C(O)R†, —C(O)OR†, —C(O)C(O)R†, —C(O)CH₂C(O)R†, S(O)₂R†, S(O)₂NR†₂, C(S)NR†₂, C(NH)NR†₂, or —N(R†)S(O)₂R†; wherein each R† is independently hydrogen, C₁₋₆ aliphatic which may be substituted as defined below, unsubstituted OPh, or an unsubstituted 5-6membered saturated, partially unsaturated, or aryl ring having 0-4 heteroatoms independently selected from nitrogen, oxygen, or sulfur, or, notwithstanding the definition above, two independent occurrences of R†, taken together with their intervening atom(s) form an unsubstituted 3-12membered saturated, partially unsaturated, or aryl mono or bicyclic ring having 0-4 heteroatoms independently selected from nitrogen, oxygen, or sulfur.

Suitable substituents on the aliphatic group of R† are independently halogen, —R●, —(halon●), —OH, —OR●, —O—(haloR●), —CN, —C(O)OH, —C(O)OR●, —NH₂, —NHR●, —NR●₂, or —NO₂, wherein each le is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

The compounds may include one or more atoms that are enriched for an isotope. For example, the compounds may have one or more hydrogen atoms replaced with deuterium or tritium. Isotopic substitution or enrichment may occur at carbon, sulfur, or phosphorus atoms as well. The compounds may be isotopically substituted or enriched for a given atom at one or more positions within the compound, or the compounds may be isotopically substituted or enriched at all instances of a given atom within the compound.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent” may be used to refer to a compound or entity of any chemical class including, for example, a polypeptide, nucleic acid, saccharide, lipid, small molecule, metal, or combination thereof. Those of ordinary skill in the art will appreciate that, in general, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer. In some embodiments, the term may refer to a compound or entity that lacks or is substantially free of any polymeric moiety

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, cattle, a primate, and/or a pig). In some embodiments, animals include, but are not limited to, mammals, birds, reptiles, amphibians, fish, and/or worms. In some embodiments, an animal may be a transgenic animal, a genetically-engineered animal, and/or a clone.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Composition: Those skilled in the art will appreciate that the term “composition” may be used to refer to a discrete physical entity that comprises one or more specified components. In general, unless otherwise specified, a composition may be of any form e.g., gas, gel, liquid, solid, etc.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Dosing regimen: Those skilled in the art will appreciate that the term “dosing regimen” may be used to refer o a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount In some embodiments, a dosing regimen is correlated with a desired or beneficial outcome when administered across a relevant population (i.e., is a therapeutic dosing regimen).

Intraperitoneal: The phrases “intraperitoneal administration” and “administered intraperitoneally” as used herein have their art-understood meaning referring to administration of a compound or composition into the peritoneum of a subject.

Oral: The phrases “oral administration” and “administered orally” as used herein have their art-understood meaning referring to administration by mouth of a compound or composition.

Parenteral: The phrases “parenteral administration” and “administered parenterally” as used herein have their art-understood meaning referring to modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrastemal injection and infusion.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. In some embodiments, a pharmaceutically acceptable salt is an alkali salt. In some embodiments, a pharmaceutically acceptable salt is a sodium salt. In some embodiments, a pharmaceutically acceptable salt is an alkaline earth metal salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Sample: A “sample” as used herein is a specific organism or material obtained therefrom. In some embodiments, a sample is a biological sample obtained or derived from a source of interest, as described herein. In some embodiments, a source of interest comprises an organism, such as an animal or human. In some embodiments, a biological sample comprises biological tissue or fluid. In some embodiments, a biological sample is or comprises bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions and/or excretions; and/or cells therefrom, etc. In some embodiments, a biological sample is or comprises cells obtained from an individual. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc. In some embodiments, a sample is an organism. In some embodiments, a sample is a plant. In some embodiments, a sample is an animal. In some embodiments, a sample is a human. In some embodiments, a sample is an organism other than a human.

Subject: As used herein, the term “subject” or “test subject” refers to any organism to which a provided compound or composition is administered in accordance with the present disclosure e.g., for experimental, diagnostic, prophylactic, and/or therapeutic purposes. Typical subjects include animals (e.g., mammals such as mice, rats, rabbits, non-human primates, and humans; insects; worms; etc.) and plants. In some embodiments, a subject may be suffering from, and/or susceptible to a disease, disorder, and/or condition.

Suffering from: An individual who is “suffering from” a disease, disorder, and/or condition has been diagnosed with and/or displays one or more symptoms of a disease, disorder, and/or condition.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Systemic: The phrases “systemic administration,” “administered systemically,” “peripheral administration,” and “administered peripherally” as used herein have their art-understood meaning referring to administration of a compound or composition such that it enters the recipient's system.

Tautomeric forms: The phrase “tautomeric forms,” as used herein, is used to describe different isomeric forms of organic compounds that are capable of facile interconversion. Tautomers may be characterized by the formal migration of a hydrogen atom or proton, accompanied by a switch of a single bond and adjacent double bond. In some embodiments, tautomers may result from prototropic tautomerism (i.e., the relocation of a proton). In some embodiments, tautomers may result from valence tautomerism (i.e., the rapid reorganization of bonding electrons). All such tautomeric forms are intended to be included within the scope of the present disclosure. In some embodiments, tautomeric forms of a compound exist in mobile equilibrium with each other, so that attempts to prepare the separate substances results in the formation of a mixture. In some embodiments, tautomeric forms of a compound are separable and isolatable compounds. In some embodiments of the disclosure, chemical compositions may be provided that are or include pure preparations of a single tautomeric form of a compound. In some embodiments, chemical compositions may be provided as mixtures of two or more tautomeric forms of a compound. In certain embodiments, such mixtures contain equal amounts of different tautomeric forms; in certain embodiments, such mixtures contain different amounts of at least two different tautomeric forms of a compound. In some embodiments of the disclosure, chemical compositions may contain all tautomeric forms of a compound. In some embodiments of the disclosure, chemical compositions may contain less than all tautomeric forms of a compound. In some embodiments of the disclosure, chemical compositions may contain one or more tautomeric forms of a compound in amounts that vary over time as a result of interconversion. In some embodiments of the disclosure, the tautomerism is keto-enol tautomerism. One of skill in the chemical arts would recognize that a keto-enol tautomer can be “trapped” (i.e., chemically modified such that it remains in the “enol” form) using any suitable reagent known in the chemical arts in to provide an enol derivative that may subsequently be isolated using one or more suitable techniques known in the art. Unless otherwise indicated, the present disclosure encompasses all tautomeric forms of relevant compounds, whether in pure form or in admixture with one another.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Therapeutically effective amount: As used herein, the term “therapeutically effective amount” means an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. In some embodiments, a therapeutically effective amount is administered in a single dose; in some embodiments, multiple unit doses are required to deliver a therapeutically effective amount.

Treat: As used herein, the term “treat,” “treatment,” or “treating” refers to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example for the purpose of decreasing the risk of developing pathology associated with the disease, disorder, and/or condition.

### 2. Detailed Description of Certain Embodiments

Short-chain fatty acids are related to various diseases, disorders, or conditions. For example, many inflammatory conditions of the gastrointestinal tract such as, for example, inflammatory bowel diseases, irritable bowel syndrome, etc. are connected with altered levels of short-chain fatty acids, often as a result of changed microbial fermentation.

Among other things, the present disclosure provides compositions and/or compounds that deliver and/or comprise short-chain fatty acid entities (e.g., short-chain fatty acid compounds, short-chain fatty acid moieties (e.g., CH₃CH₂CH₂COO— in compound I-1), etc.). In some embodiments, provided technologies address certain long-felt needs, particularly those related to administration of short-chain fatty acid entities to humans. For example, although beneficial impacts of short-chain fatty acids on gastrointestinal health have been reported for many years, in many cases clinical benefits through administration of short-chain fatty acids themselves or salts thereof have not established. For example reports, see van der Beek, et al., Role of short-chain fatty acids in colonic inflammation, carcinogenesis, and mucosal protection and healing, Nutrition Reviews Vol. 75(4):286-305.

Among other things, the present disclosure identifies the source(s) of certain problems encountered in prior efforts to effectively administer short-chain fatty acid entities, particularly to humans, to deliver clinical benefits. In some embodiments, the present disclosure appreciates that prior technologies may not have been able to administer such entities at high enough quantities to be efficacious. In some embodiments, the present disclosure appreciates that certain compositions cannot be readily formulated for administration to subjects, for example, because of their high viscosity. Additionally or alternatively, in some embodiments, the present disclosure observes that certain compositions, for example, those having free carboxylic acid groups, can be very unpalatable, rendering oral administration difficult if not impossible. The present disclosure recognizes that prior technologies have typically administered short-chain fatty acids in relatively low dosages, via limited administration methods, and/or in particular forms or formats; for example, short chain fatty acids were often administered as free acids (or salt) via enema, which can cause significant inconvenience and discomfort to patients.

Among other things, the present disclosure provides technologies that can effectively deliver short-chain fatty acid entities. In some embodiments, through provided compounds, e.g., those of formula I, the present disclosure provides compounds and compositions of greatly improved properties and/or activities, and/or low toxicity, which can be delivered efficiently through various administration methods and dosing regimens, including those of high unit doses and/or total doses, if such high dosing regimens are desirable. In some embodiments, the present disclosure provides new and/or more effective therapeutics for certain conditions, disorders and/or diseases, e.g., inflammatory conditions of the gastrointestinal tract, such as inflammatory bowel diseases, irritable bowel syndrome, etc. In some embodiments, provided compounds have optimized flow properties so that they can easily formulated and administered to subjects through, e.g., oral administration. In some embodiments, provided compounds are palatable and can be formulated for oral formulation. In some embodiments, provided compounds have sufficient flow properties and are palatable, and can be administered orally by drinking provided compounds or pharmaceutically acceptable liquid formulations (e.g., solution, suspension, etc.) thereof. In some embodiments, provided compounds are of low toxicity and can be administered in high quantities, e.g., at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4 or 5 g/kg per day.

In some embodiments, a provided compound has the structure of formula I:

R^{A}-L-R^{B}, I

or a salt thereof, wherein:
R^{A} is

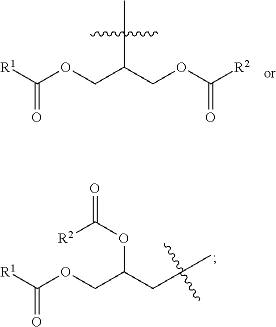

R^{B} is

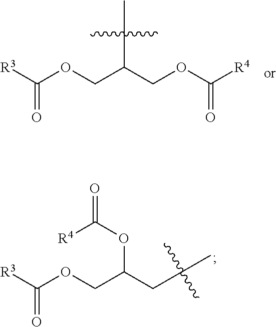

each of R¹, R², R³ and R⁴ is independently C₁₋₃ alkyl;
L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with bivalent C₁₄ aliphatic, —O—, —C(O)—, —C(O)O—, —S—, —N(R′)—, or —C(O)N(R′)—;
R′ is R or —C(O)R;
each R is independently hydrogen, or an optionally substituted group selected from C₁₋₆ aliphatic, C₁₋₆ heteroaliphatic having 1-5 hetereoatoms independently selected from nitrogen, oxygen, and sulfur, phenyl, 5-6 membered heteroaryl having 1-5 hetereoatoms independently selected from nitrogen, oxygen, and sulfur, and 3-6 membered heterocyclyl having 1-5 hetereoatoms independently selected from nitrogen, oxygen, and sulfur.

In some embodiments, L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with bivalent C₁₋₄ aliphatic, —O—, —C(O)—, —C(O)O—, —S—, —N(R′)—, or —C(O)N(R′)—, wherein each R′ is independently as described in the present disclosure. In some embodiments, L is an optionally substituted, linear or branched, bivalent group selected from C₂₋₆ aliphatic and C₂₋₆ heteroaliphatic having 1-5 hetereoatoms independently selected from nitrogen, oxygen, and sulfur, wherein two or more methylene units of the aliphatic and heteroaliphatic are optionally and independently replaced with bivalent C₁₋₄ aliphatic, —O—, —C(O)—, —C(O)O—, —S—, —N(R′)—, or —C(O)N(R′)—, wherein each R′ is independently as described in the present disclosure. In some embodiments, L is an optionally substituted, linear or branched, bivalent group selected from C₂₋₆ aliphatic and C₂₋₆ heteroaliphatic having 1-5 hetereoatoms independently selected from nitrogen, oxygen, and sulfur, wherein two methylene units of the aliphatic and heteroaliphatic are optionally and independently replaced with bivalent C₁₋₄ aliphatic, —O—, —C(O)—, —C(O)O—, S, —N(R′)—, or —C(O)N(R′)—, wherein each R′ is independently as described in the present disclosure. In some embodiments, L is an optionally substituted, linear or branched, bivalent group selected from C₂₋₆ aliphatic and C₂₋₆ heteroaliphatic having 1-5 hetereoatoms independently selected from nitrogen, oxygen, and sulfur, wherein the two methylene units, directly bonded to R^{A} and R^{B}, of the aliphatic and heteroaliphatic, are optionally and independently replaced with bivalent C₁₋₄ aliphatic, —O—, —C(O)—, —C(O)O—, —S—, —N(R′)—, or C(O)N(R′)—, wherein each R′ is independently as described in the present disclosure. In some embodiments, L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with —O—, —C(O)—, —C(O)O—, N(R′)—, or —C(O)N(R′)—, wherein each R′ is independently as described in the present disclosure. In some embodiments, L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with —O—. In some embodiments, L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with —C(O)O—. In some embodiments, L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with —N(R′)—, wherein each R′ is independently as described in the present disclosure. In some embodiments, L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with —NH—. In some embodiments, L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with —C(O)N(R′)—, wherein each R′ is independently as described in the present disclosure. In some embodiments, L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with —C(O)NH—.

In some embodiments, L is an optionally substituted, linear or branched, bivalent C₁₋₆ aliphatic, wherein one or more methylene units of the aliphatic are optionally and independently replaced with bivalent C₁₋₄ aliphatic, —O—, —C(O)—, —C(O)O—, —S—, —N(R′)—, or —C(O)N(R′)—, wherein each R′ is independently as described in the present disclosure. In some embodiments, L is an optionally substituted, linear or branched, bivalent C₂₋₆ aliphatic, wherein two or more methylene units of the aliphatic are optionally and independently replaced with bivalent C₁₋₄ aliphatic, —O—, —C(O)—, —C(O)O—, —S—, —N(R′)—, or —C(O)N(R′)—, wherein each R′ is independently as described in the present disclosure. In some embodiments, L is an optionally substituted, linear or branched, bivalent C₂₋₆ aliphatic, wherein two methylene units of the aliphatic are optionally and independently replaced with bivalent C₁₋₄ aliphatic, —O—, —C(O)—, —C(O)O—, —S—, —N(R′)—, or —C(O)N(R′)—, wherein each R′ is independently as described in the present disclosure. In some embodiments, L is an optionally substituted, linear or branched, bivalent C₂₋₆ aliphatic, wherein the two methylene directly bonded to R^{A} and R^{B} are optionally and independently replaced with bivalent C₁₋₄ aliphatic, —O—, —C(O)—, —C(O)O—, —S—, —N(R′)—, or —C(O)N(R′)—, wherein each R′ is independently as described in the present disclosure. In some embodiments, L is an optionally substituted, linear or branched, bivalent aliphatic, wherein one or more methylene units of the aliphatic are optionally and independently replaced with —O—, —C(O)—, —C(O)O—, —N(R′)—, or —C(O)N(R′)—, wherein each R′ is independently as described in the present disclosure. In some embodiments, L is an optionally substituted, linear or branched, bivalent aliphatic, wherein one or more methylene units of the aliphatic are optionally and independently replaced with —O—. In some embodiments, L is an optionally substituted, linear or branched, bivalent aliphatic, wherein one or more methylene units of the aliphatic are optionally and independently replaced with —C(O)O—. In some embodiments, L is an optionally substituted, linear or branched, bivalent aliphatic, wherein one or more methylene units of the aliphatic are optionally and independently replaced with N(R′)—, wherein each R′ is independently as described in the present disclosure. In some embodiments, L is an optionally substituted, linear or branched, bivalent aliphatic, wherein one or more methylene units of the aliphatic are optionally and independently replaced with —NH—. In some embodiments, L is an optionally substituted, linear or branched, bivalent aliphatic, wherein one or more methylene units of the aliphatic are optionally and independently replaced with —C(O)N(R′)—, wherein each R′ is independently as described in the present disclosure. In some embodiments, L is an optionally substituted, linear or branched, bivalent aliphatic, wherein one or more methylene units of the aliphatic are optionally and independently replaced with —C(O)NH—.

In some embodiments, L is L¹-L²-L³, wherein:
each of L¹, L², and L³ is independently —O—, —C(O)O—, —S—, —N(R′)—or —C(O)N(R′)—; and
L² is an optionally substituted, linear or branched, bivalent group selected from C₁₋₄ aliphatic and C₁₋₄ heteroaliphatic having 1-3 hetereoatoms independently selected from nitrogen, oxygen, and sulfur, wherein one or more methylene units of the aliphatic and heteroaliphatic are optionally and independently replaced with bivalent C₁₄ aliphatic, —O—, —C(O)—, —C(O)O—, —S—, —N(R′)—, or —C(O)N(R′)—;
wherein each R′ is independently as described in the present disclosure.

In some embodiments, L¹ and L³ are the same. In some embodiments, L¹ and L³ are different.

In some embodiments, L¹ is —O—. In some embodiments, L¹ is —C(O)O—. In some embodiments, L¹ is S. In some embodiments, L¹ is —N(R′)—, wherein R′ is as described in the present disclosure. In some embodiments, L¹ is —NH—. In some embodiments, L¹ is —C(O)N(R′)—, wherein R′ is as described in the present disclosure. In some embodiments, L¹ is —C(O)NH—.

In some embodiments, L² is an optionally substituted, linear or branched, bivalent group selected from C₁₋₄ aliphatic and C₁₋₄ heteroaliphatic having 1-3 hetereoatoms independently selected from nitrogen, oxygen, and sulfur, wherein one or more methylene units of the aliphatic and heteroaliphatic are optionally and independently replaced with bivalent C₁₋₄ aliphatic, —O—, —C(O)—, —C(O)O—, —S—, —N(R′)—, or —C(O)N(R′)—, wherein each R′ is independently as described in the present disclosure. In some embodiments, L² is an optionally substituted, linear or branched, bivalent C₁₋₄ aliphatic, wherein one or more methylene units of the aliphatic and heteroaliphatic are optionally and independently replaced with bivalent C₁₋₄ aliphatic, —O—, —C(O)—, —C(O)O—, —S—, —N(R′)—, or —C(O)N(R′)—, wherein each R′ is independently as described in the present disclosure. In some embodiments, L² is optionally substituted bivalent C₁₋₄ aliphatic. In some embodiments, L² is optionally substituted C₁₋₄ alkylene. In some embodiments, L² is optionally substituted —CH₂—. In some embodiments, L² is unsubstituted —CH₂. In some embodiments, L² is substituted —CH₂—. In some embodiments, L² is optionally substituted —CH₂CH₂—. In some embodiments, L² is unsubstituted —CH₂CH₂—. In some embodiments, L² is substituted —CH₂CH₂—. In some embodiments, L² is optionally substituted —CH₂CH₂CH₂—. In some embodiments, L² is unsubstituted —CH₂CH₂CH₂—. In some embodiments, L² is substituted —CH₂CH₂CH₂—. In some embodiments, L² is optionally substituted —CH₂CH₂CH₂CH₂—. In some embodiments, L² is unsubstituted —CH₂CH₂CH₂CH₂—. In some embodiments, L² is substituted —CH₂CH₂CH₂CH₂—.

In some embodiments, L³ is —O—. In some embodiments, L³ is —C(O)O—. In some embodiments, L³ is S. In some embodiments, L³ is —N(R′)—, wherein R′ is as described in the present disclosure. In some embodiments, L³ is —NH—. In some embodiments, L³ is —C(O)N(R′)—, wherein R′ is as described in the present disclosure. In some embodiments, L³ is —C(O)NH—.

In some embodiments, both L¹ and L³ are —O—. In some embodiments, both L¹ and L³ are —C(O)O—. In some embodiments, both L¹ and L³ are —C(O)O—, wherein R^{A} and R^{B} are bonded to —O—. In some embodiments, both L¹ and L³ are —S—. In some embodiments, both L¹ and L³ are —N(R′)—, wherein each R′ is independently as described in the present disclosure. In some embodiments, both L¹ and L³ are the same and are —N(R′)—, wherein each R′ is independently as described in the present disclosure. In some embodiments, both L¹ and L³ are —NH—. In some embodiments, both L¹ and L³ are —C(O)N(R′)—, wherein each R′ is independently as described in the present disclosure. In some embodiments, both L¹ and L³ are the same and are —C(O)N(R′)—, wherein each R′ is independently as described in the present disclosure. In some embodiments, both L¹ and L³ are —C(O)NH—. In some embodiments, both R^{A} and R^{B} are bonded to —NH—.

In some embodiments, L is a dicarboxyl group, e.g., —OC(O)—L²—C(O)O—, wherein L² is as described in the present disclosure. In some embodiments, L is a dicarboxyl group of a TCA cycle di- or tri-acid, e.g., succinic acid. In some embodiments, L is —OC(O)—CH₂CH₂—C(O)O—.

In some embodiments, a provided compound has the structure of formula I-a or a salt thereof:

R^{A}-L¹-L²-L³-R^{B}, I-a

wherein each variable is independently as described in the present disclosure.

In some embodiments, a provided compound has the structure of formula I-b or a salt thereof:

R^{A}—O(O)C—CH₂CH₂—C(O)O—R^{B},

wherein each variable is independently as described in the present disclosure.

In some embodiments, R^{A} and R^{B} are different. In some embodiments, one of R¹ and R² is the same as one of R³ and R⁴, and the other of R¹ and R² is the same as the other of R³ and R⁴. In some embodiments, R¹ is the same as R³. In some embodiments, R² is the same as R⁴. In some embodiments, R¹ is the same as R³, and R² is the same as R⁴.

In some embodiments, R^{A} and R^{B} are the same.

In some embodiments, R^{A} and R^{B} each independently comprise a chiral center. In some embodiments, one of R^{A} and R^{B} is symmetric and the other one is not. In some embodiments, both R^{A} and R^{B} are symmetric. In some embodiments, R^{A} comprises a chiral center and R^{B} comprises no chiral centers. In some embodiments, R^{A} comprises no chiral centers and R^{B} comprises a chiral center.

In some embodiments, R^{A} is

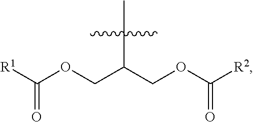

wherein each variable is independently as described in the present disclosure. In some embodiments, R^{A} is

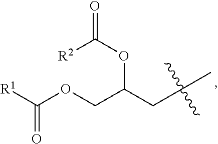

wherein each variable is independently as described in the present disclosure. In some embodiments, R^{B} is

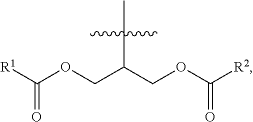

wherein each variable is independently as described in the present disclosure. In some embodiments, R^{B} is

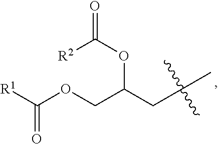

wherein each variable is independently as described in the present disclosure.

In some embodiments, R^{A} is

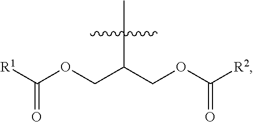

### and R^{B} is

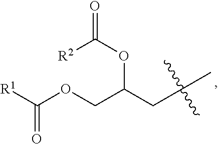

wherein each variable is independently as described in the present disclosure. In some embodiments, each of R^{A} and R^{B} is independently

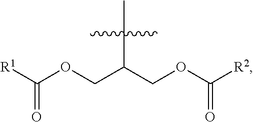

wherein each variable is independently as described in the present disclosure. In some embodiments, R^{A} and R^{B} are the same and are

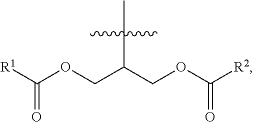

wherein each variable is independently as described in the present disclosure. In some embodiments, each of R^{A} and R^{B} is independently

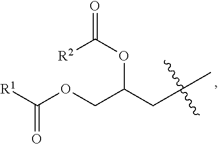

wherein each variable is independently as described in the present disclosure. In some embodiments, R^{A} and R^{B} are the same and are

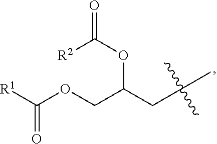

wherein each variable is independently as described in the present disclosure. In some embodiments, R^{A} is

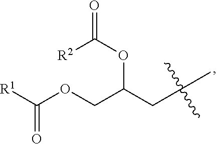

### and R^{B} is

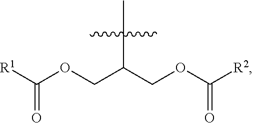

wherein each variable is independently as described in the present disclosure.

In some embodiments, a provided compound has the structure of formula I-c or a pharmaceutically acceptable salt thereof:

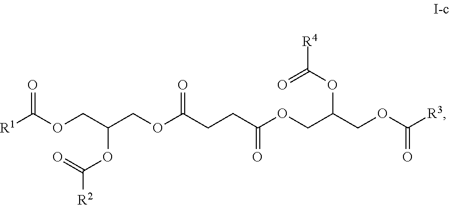

wherein each variable is independently as described in the present disclosure.

In some embodiments, a provided compound has the structure of formula I-d or a pharmaceutically acceptable salt thereof:

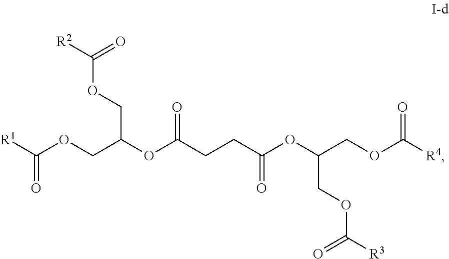

wherein each variable is independently as described in the present disclosure.

In some embodiments, a provided compound has the structure of formula I-e or a pharmaceutically acceptable salt thereof:

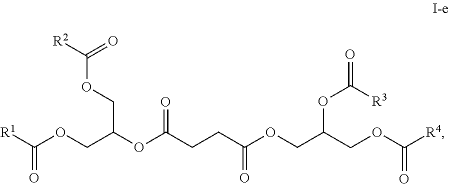

wherein each variable is independently as described in the present disclosure.

In some embodiments, R¹ is methyl. In some embodiments, R¹ is ethyl. In some embodiments, R¹ is propyl. In some embodiments, R¹ is n-propyl. In some embodiments, R¹ is selected from methyl, ethyl, and n-propyl.

In some embodiments, R² is methyl. In some embodiments, R² is ethyl. In some embodiments, R² is propyl. In some embodiments, R² is n-propyl. In some embodiments, R² is selected from methyl, ethyl, and n-propyl.

In some embodiments, R³ is methyl. In some embodiments, R³ is ethyl. In some embodiments, R³ is propyl. In some embodiments, R³ is n-propyl. In some embodiments, R³ is selected from methyl, ethyl, and n-propyl.

In some embodiments, R⁴ is methyl. In some embodiments, R⁴ is ethyl. In some embodiments, R⁴ is propyl. In some embodiments, R⁴ is n-propyl. In some embodiments, R⁴ is selected from methyl, ethyl, and n-propyl.

In some embodiments, R¹, R², R³ and R⁴ are the same. In some embodiments, at least one of R¹, R², R³ and R⁴ are different from at least another R¹, R², R³ and R⁴. In some embodiments, two of _{R}1, R2, _{R}³ and R⁴ are the same. In some embodiments, three of R¹, R², R³ and R⁴ are the same. In some embodiments, all of R¹, R², R³ and R⁴ are the same.

In some embodiments, one of R¹, R², R³ and R⁴ is methyl. In some embodiments, one of R¹, R², R³ and R⁴ is ethyl. In some embodiments, one of R¹, R², R³ and R⁴ is propyl. In some embodiments, one or R¹, R², R³ and R⁴ is n-propyl.

In some embodiments, each of R¹, R², R³ and R⁴ is n-propyl.

In some embodiments, a provided compound is compound I-I:

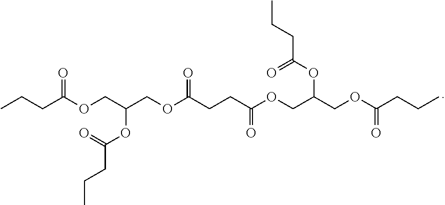 [SMILES: CCCC(=O)OCC(COC(=O)CCC(=O)OCC(COC(=O)CCC)OC(=O)CCC)OC(=O)CCC]

In some embodiments, a provided compound is compound 1-2:

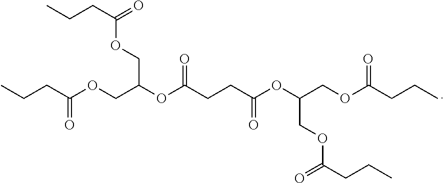 [SMILES: CCCC(=O)OCC(COC(=O)CCC)OC(=O)CCC(=O)OC(COC(=O)CCC)COC(=O)CCC]

In some embodiments, once administered, provided compounds are hydrolyzed, with and/or without involvement of an enzyme, to release one or more short-chain fatty acids (and/or salts thereof). In some embodiments, provided compounds, after administration, can be hydrolyzed to release four moles of short-chain fatty acids (and/or salts thereof) per mole of administered provided compounds. In some embodiments, at a human physiological pH, short-chain fatty acids may exist as salts thereof, for example, at pH around 7.4, large portions of short-chain fatty acids exist as their salts.

In some embodiments, provided compounds can be hydrolyzed to provide compounds including R¹—COOH, R²—COOH, R³—COOH, R⁴—COOH, H-L-H, and salts thereof, wherein each variable is independently as described in the present disclosure. In some embodiments, provided compounds can be hydrolyzed to provide compounds including R¹—COOH, R²—COOH, R³—COOH, R⁴—COOH, HOC(O)-L²-C(O)OH, and salts thereof, wherein each variable is independently as described in the present disclosure. In some embodiments, each hydrolysis product is independently selected from R¹—COOH, R²—COOH, R³—COOH, R⁴—COOH, H-L-H, and salts thereof, and glycerol, wherein each variable is independently as described in the present disclosure. In some embodiments, each hydrolysis product is independently selected from R¹—COOH, R²—COOH, R³—COOH, R⁴—COOH, HOC(O)-L²-C(O)OH, and salts thereof, and glycerol, wherein each variable is independently as described in the present disclosure.

In some embodiments, R′ is R, wherein R is as described in the present disclosure. In some embodiments, R′ is —C(O)R, wherein R is as described in the present disclosure. In some embodiments, R′ is —H.

In some embodiments, R is hydrogen. In some embodiments, R is an optionally substituted group selected from C₁₋₆ aliphatic, C₁₋₆ heteroaliphatic having 1-5 hetereoatoms independently selected from nitrogen, oxygen, and sulfur, phenyl, 5-6 membered heteroaryl having 1-5 hetereoatoms independently selected from nitrogen, oxygen, and sulfur, and 3-6 membered heterocyclyl having 1-5 hetereoatoms independently selected from nitrogen, oxygen, and sulfur.

In some embodiments, R is substituted. In some embodiments, R is unsubstituted.

In some embodiments, R is optionally substituted C₁₋₆ aliphatic. In some embodiments, R is optionally substituted C₁₋₆ alkyl. In some embodiments, R is optionally substituted methyl. In some embodiments, R is optionally substituted ethyl. In some embodiments, R is optionally substituted propyl. In some embodiments, R is optionally substituted butyl. In some embodiments, R is optionally substituted pentyl. In some embodiments, R is optionally substituted hexyl. In some embodiments, R is optionally substituted C₁₋₆ cycloaliphatic. In some embodiments, R is optionally substituted C₁₋₆ alkyl. In some embodiments, R is optionally substituted cyclpropyl. In some embodiments, R is optionally substituted cyclobutyl. In some embodiments, R is optionally substituted cyclopentyl. In some embodiments, R is optionally substituted cyclohexyl.

In some embodiments, R is optionally substituted C₁₋₆ heteroaliphatic having 1-5 hetereoatoms independently selected from nitrogen, oxygen, and sulfur.

In some embodiments, R is optionally substituted phenyl. In some embodiments, R is phenyl. In some embodiments, R is substituted phenyl.

In some embodiments, R is optionally substituted 5-6 membered heteroaryl having 1-5 hetereoatoms independently selected from nitrogen, oxygen, and sulfur. In some embodiments, R is optionally substituted 5-membered heteroaryl having 1-5 hetereoatoms independently selected from nitrogen, oxygen, and sulfur. In some embodiments, R is optionally substituted 6-membered heteroaryl having 1-5 hetereoatoms independently selected from nitrogen, oxygen, and sulfur.

In some embodiments, R is optionally substituted 3-6 membered heterocyclyl having 1-5 hetereoatoms independently selected from nitrogen, oxygen, and sulfur. In some embodiments, R is optionally substituted 3-membered heterocyclyl having 1-5 hetereoatoms independently selected from nitrogen, oxygen, and sulfur. In some embodiments, R is optionally substituted 4-membered heterocyclyl having 1-5 hetereoatoms independently selected from nitrogen, oxygen, and sulfur. In some embodiments, R is optionally substituted 5-membered heterocyclyl having 1-5 hetereoatoms independently selected from nitrogen, oxygen, and sulfur. In some embodiments, R is optionally substituted 6-membered heterocyclyl having 1-5 hetereoatoms independently selected from nitrogen, oxygen, and sulfur.

In some embodiments, as illustrated in compounds I-1 and 1-2, each hydrolysis product is independently a short-chain fatty acid (or a salt thereof), glycerol, or a TCA cycle acid (or a salt thereof). In some embodiments, each hydrolysis product is independently a natural product. In some embodiments, each hydrolysis product is of very low toxicity, for example, short-chain fatty acids (or salts thereof), glycerol, or TCA cycle acids (or salts thereof). Thus, among other things, the present disclosure provides compounds of low toxicity and can be administered at high levels, for example, at large unit dose and/or total dose.

In some embodiments, a provided compound has a purity of 60%-100%. In some embodiments, a provided compound has a purity of at least 60%, 65%, 70%, 75%, 80%, 85%, 90%, 91%, 92%, 93%, 94%, 95%, 96%, 97%, 98%, or 99%. In some embodiments, a provided compound has a purity of at least 60%. In some embodiments, a provided compound has a purity of at least 70%. In some embodiments, a provided compound has a purity of at least 80%. In some embodiments, a provided compound has a purity of at least 85%. In some embodiments, a provided compound has a purity of at least 90%. In some embodiments, a provided compound has a purity of at least 91%. In some embodiments, a provided compound has a purity of at least 92%. In some embodiments, a provided compound has a purity of at least 93%. In some embodiments, a provided compound has a purity of at least 94%. In some embodiments, a provided compound has a purity of at least 95%. In some embodiments, a provided compound has a purity of at least 96%. In some embodiments, a provided compound has a purity of at least 97%. In some embodiments, a provided compound has a purity of at least 98%. In some embodiments, a provided compound has a purity of at least 99%. In some embodiments, a provided compound has a purity of at least 99.5%.

### Compositions

In some embodiments, the present disclosure provides compositions comprising provided compounds. In some embodiments, a provided composition is a pharmaceutical composition, comprising a provided compound or a pharmaceutically salt thereof, and optionally a pharmaceutically acceptable carrier. As described herein, provided compounds can have greatly improved physical and/or chemical properties that greatly facilitate formulation processes. In some embodiments, provided compounds can be directly administered without any pharmaceutical carriers. In some embodiments, a provided pharmaceutical composition consists of a provided compound. In some embodiments, a composition consisting of a provided compound is of sufficiently low viscosity and acceptable taste so that it can be directly administered by drinking.

In some embodiments, the present disclosure provides pharmaceutical compositions. In some embodiments, a provided pharmaceutical composition comprises a therapeutically effective amount of a provided compound, and optionally a pharmaceutically acceptable inactive ingredient selected from pharmaceutically acceptable diluents, pharmaceutically acceptable excipients, and pharmaceutically acceptable carriers. In some embodiments, a pharmaceutical composition is formulated for intravenous injection, oral administration, buccal administration, inhalation, nasal administration, topical administration, ophthalmic administration or otic administration. In some embodiments, a provided composition is for oral administration. In some embodiments, a provided composition is for oral administration by direct drinking. In some embodiments, a provided composition is for administration by enema. In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

Among other things, the present disclosure recognizes that properties of provided compounds and/or compositions, such as flow properties and/or taste, etc., are important for pharmaceutical formulations. For example, in some embodiments, a compound may be too viscous for formulation, and/or too unpalatable (e.g., bitter) for oral administration and/or good patient compliance. In some embodiments, provided technologies, for example, those with butyric acid and/or caprylic acid moieties, and/or without free succinic acid -COOH groups, and/or without free carboxylic acid and/or hydroxyl groups, such as compounds I-1 and I-2, were tested to have surprisingly good flow properties and taste for formulation. In some embodiments, provided compounds can be administered by direct oral administration. In some embodiments, provided compounds have suitable flow property and taste and can be administered by direct drinking by a subject. In some embodiments, viscosity of a provided liquid compound or a liquid composition is no more than 0.9, 1, 2, 3, 4, 5, 6, 7, 8, 9, 10, 20, 30, 40, 50, 60, 70, 80, 90, 100, 200, 300, 400, 500, 600, 700, 800, 900, 1000, 1100, 1200, 1500 or 2000 cP at a temperature. In some embodiments, a temperature is 25° C. In some embodiments, a temperature is 20° C. In some embodiments, a temperature is room temperature. In some embodiments, the viscosity is no more than 1500 cP at a temperature. In some embodiments, the viscosity is no more than 1200 cP at a temperature. In some embodiments, the viscosity is no more than 1000 cP at a temperature. In some embodiments, the viscosity is no more than 900 cP at a temperature. In some embodiments, the viscosity is no more than 800 cP at a temperature. In some embodiments, the viscosity is no more than 700 cP at a temperature. In some embodiments, the viscosity is no more than 600 cP at a temperature. In some embodiments, the viscosity is no more than 500 cP at a temperature. In some embodiments, the viscosity is no more than 400 cP at a temperature. In some embodiments, the viscosity is no more than 300 cP at a temperature. In some embodiments, the viscosity is no more than 200 cP at a temperature. In some embodiments, the viscosity is no more than 100 cP at a temperature. In some embodiments, the viscosity is no more than 50 cP at a temperature. In some embodiments, the viscosity is no more than 40 cP at a temperature. In some embodiments, the viscosity is no more than 30 cP at a temperature. In some embodiments, the viscosity is no more than 20 cP at a temperature. In some embodiments, the viscosity is no more than 10 cP at a temperature. In some embodiments, the viscosity is no more than 9 cP at a temperature. In some embodiments, the viscosity is no more than 8 cP at a temperature. In some embodiments, the viscosity is no more than 7 cP at a temperature. In some embodiments, the viscosity is no more than 6 cP at a temperature. In some embodiments, the viscosity is no more than 5 cP at a temperature. In some embodiments, the viscosity is no more than 4 cP at a temperature. In some embodiments, the viscosity is no more than 3 cP at a temperature. In some embodiments, the viscosity is no more than 2 cP at a temperature. In some embodiments, the viscosity is no more than 1 cP at a temperature. In some embodiments, a temperature is 25° C. In some embodiments, a temperature is 20° C. In some embodiments, a temperature is room temperature. In some embodiments, provided compounds are less viscous than glycerol at a temperature, e.g., room temperature. In some embodiments, certain compounds can be administered, e.g., by direct drinking, in large quantities.

In some embodiments, provided compounds, e.g., compounds I-1 and 1-2, are administered in large quantities to provide or improve efficacy (for example, without any intent to be limited by theory, as a result of their improved flow properties, taste, and/or low toxicity). In some embodiments, a quantity is at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4 or 5 g/kg. In some embodiments, a quantity is at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4 or 5 g/kg per day. In some embodiments, a quantity is at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4 or 5 g/kg per single dose. In some embodiments, a quantity is at least 0.1 g/kg. In some embodiments, a quantity is at least 0.1 g/kg. In some embodiments, a quantity is at least 0.2 g/kg. In some embodiments, a quantity is at least 0.3 g/kg. In some embodiments, a quantity is at least 0.4 g/kg. In some embodiments, a quantity is at least 0.5 g/kg. In some embodiments, a quantity is at least 0.6 g/kg. In some embodiments, a quantity is at least 0.7 g/kg. In some embodiments, a quantity is at least 0.8 g/kg. In some embodiments, a quantity is at least 0.9 g/kg. In some embodiments, a quantity is at least 1 g/kg. In some embodiments, a quantity is at least 1.1 g/kg. In some embodiments, a quantity is at least 1.2 g/kg. In some embodiments, a quantity is at least 1.3 g/kg. In some embodiments, a quantity is at least 1.4 g/kg. In some embodiments, a quantity is at least 1.5 g/kg. In some embodiments, a quantity is at least 1.6 g/kg. In some embodiments, a quantity is at least 1.7 g/kg. In some embodiments, a quantity is at least 1.8 g/kg. In some embodiments, a quantity is at least 1.9 g/kg. In some embodiments, a quantity is at least 2 g/kg. In some embodiments, a quantity is at least 2.5 g/kg. In some embodiments, a quantity is at least 3 g/kg. In some embodiments, a quantity is at least 3.5 g/kg. In some embodiments, a quantity is at least 4 g/kg. In some embodiments, a quantity is at least 5 g/kg. In some embodiments, a quantity is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4 or 5 g/kg. In some embodiments, a quantity is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4 or 5 g/kg per day. In some embodiments, a quantity is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4 or 5 g/kg per single dose. In some embodiments, a quantity is about 0.1 g/kg. In some embodiments, a quantity is about 0.1 g/kg. In some embodiments, a quantity is about 0.2 g/kg. In some embodiments, a quantity is about 0.3 g/kg. In some embodiments, a quantity is about 0.4 g/kg. In some embodiments, a quantity is about 0.5 g/kg. In some embodiments, a quantity is about 0.6 g/kg. In some embodiments, a quantity is about 0.7 g/kg. In some embodiments, a quantity is about 0.8 g/kg. In some embodiments, a quantity is about 0.9 g/kg. In some embodiments, a quantity is about 1 g/kg. In some embodiments, a quantity is about 1.1 g/kg. In some embodiments, a quantity is about 1.2 g/kg. In some embodiments, a quantity is about 1.3 g/kg. In some embodiments, a quantity is about 1.4 g/kg. In some embodiments, a quantity is about 1.5 g/kg. In some embodiments, a quantity is about 1.6 g/kg. In some embodiments, a quantity is about 1.7 g/kg. In some embodiments, a quantity is about 1.8 g/kg. In some embodiments, a quantity is about 1.9 g/kg. In some embodiments, a quantity is about 2 g/kg. In some embodiments, a quantity is about 2.5 g/kg. In some embodiments, a quantity is about 3 g/kg. In some embodiments, a quantity is about 3.5 g/kg. In some embodiments, a quantity is about 4 g/kg. In some embodiments, a quantity is about 5 g/kg. In some embodiments, a quantity of about or at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.5, 3, 3.5, 4 or 5 g of a provided compound is administered, e.g., to a subject. In some embodiments, a quantity of about or at least 1.6, 1.7, 1.8, 1.9, 2.0, 2.5, 3, 3.5, 4 or 5 g of a provided compound is administered. In some embodiments, a quantity is about 0.5 g. In some embodiments, a quantity is about 1 g. In some embodiments, a quantity is about 1.5 g. In some embodiments, a quantity is about 1.6 g. In some embodiments, a quantity is about 1.7 g. In some embodiments, a quantity is about 1.8 g. In some embodiments, a quantity is about 1.7 g. In some embodiments, a quantity is about 1.9 g. In some embodiments, a quantity is about 2.0 g. In some embodiments, a quantity is about 2.5 g. In some embodiments, a quantity is about 3 g. In some embodiments, a quantity is about 3.5 g. In some embodiments, a quantity is about 4 g. In some embodiments, a quantity is about 5 g. In some embodiments, a quantity is at least 0.5 g. In some embodiments, a quantity is at least 1 g. In some embodiments, a quantity is at least 1.5 g. In some embodiments, a quantity is at least 1.6 g. In some embodiments, a quantity is at least 1.7 g. In some embodiments, a quantity is at least 1.8 g. In some embodiments, a quantity is at least 1.7 g. In some embodiments, a quantity is at least 1.9 g. In some embodiments, a quantity is at least 2.0 g. In some embodiments, a quantity is at least 2.5 g. In some embodiments, a quantity is at least 3 g. In some embodiments, a quantity is at least 3.5 g. In some embodiments, a quantity is at least 4 g. In some embodiments, a quantity is at least 5 g. In some embodiments, a quantity as described in the present disclosure is a per day quantity for a subject. In some embodiments, the present disclosure can thus provide significantly higher dosing (e.g., as a result of low viscosity, low toxicity, etc., of provided compounds and compositions) than prior dosing regimens, for example, those typically administering 0.3-1.0 gram of butyric acid or a salt thereof. The present disclosure, among other things, can thus significantly enhance previously reported benefits of short-chain fatty acids, and/or translate certain observed benefits into clinically significant results through, for example, various beneficial properties and/or activities of provided compounds, dosing regimens that can safely deliver large quantities of provided compounds, etc.

In therapeutic and/or diagnostic applications, provide compounds can be formulated for a variety of modes of administration, including systemic and topical or localized administration. Techniques and formulations generally may be found in Remington, The Science and Practice of Pharmacy, (20th ed. 2000).

Provided compounds and compositions thereof are effective over a wide dosage range. For example, in the treatment of adult humans, dosages from about 0.01 to about 10000 mg, from about 0.01 to about 1000 mg, from about 0.5 to about 100 mg, from about 1 to about 50 mg per day, and from about 5 to about 100 mg per day are examples of dosages that may be used. The exact dosage will depend upon the route of administration, the form in which the compound is administered, the subject to be treated, the body weight of the subject to be treated, and the preference and experience of the attending physician.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

In some embodiments, pharmaceutically acceptable salts are metal salts. In some embodiments, pharmaceutically acceptable salts are alkaline metal salts. In some embodiments, pharmaceutically acceptable salts are alkaline earth metal salts. In some embodiments, a pharmaceutically acceptable salt is a lithium, sodium, potassium, magnesium, or calcium salt. In some embodiments, a pharmaceutically acceptable salt is a sodium salt.

Depending on the specific conditions, disorders or diseases being treated, provided agents may be formulated into liquid or solid dosage forms and administered systemically or locally. The agents may be delivered, for example, in a timed- or sustained-low release form as is known to those skilled in the art. Techniques for formulation and administration may be found in Remington, The Science and Practice of Pharmacy (20th ed. 2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, provided agents may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate provided compounds or compositions into dosages suitable for systemic administration is within the scope of the disclosure. With proper choice of carrier and suitable manufacturing practice, the compositions of the present disclosure, in particular, those formulated as solutions, may be administered parenterally, such as by intravenous injection.

The compounds can be formulated readily using pharmaceutically acceptable carriers well known in the art into dosages suitable for oral administration. Such carriers enable provided compounds and compositions to be formulated as tablets, pills, capsules, liquids, gels, syrups, slurries, suspensions and the like, for oral ingestion by a subject (e.g., patient) to be treated.

For nasal or inhalation delivery, provided compounds or compositions may also be formulated by methods known to those of skill in the art, and may include, for example, but not limited to, examples of solubilizing, diluting, or dispersing substances such as, saline, preservatives, such as benzyl alcohol, absorption promoters, and fluorocarbons.

In certain embodiments, parenteral administration is by injection, by, e.g., a syringe, a pump, etc. In certain embodiments, the injection is a bolus injection. In certain embodiments, the injection is administered directly to a tissue, such as striatum, caudate, cortex, hippocampus and cerebellum.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

In some embodiments, cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGS). In addition, stabilizers may be added.

In some embodiments, provided compounds are formulated as liquids for oral administration by drinking. In some embodiments, provided compounds are liquid at room temperature, and are administered as pure compounds orally by drinking.

Depending upon the particular condition, or disease state, to be treated or prevented, additional therapeutic agents, which are normally administered to treat or prevent that condition, may be administered together with provided compounds or compositions. For example, chemotherapeutic agents or other anti-proliferative agents may be combined with provided compounds or compositions to treat proliferative diseases and cancer. Examples of known chemotherapeutic agents include, but are not limited to, adriamycin, dexamethasone, vincristine, cyclophosphamide, fluorouracil, topotecan, taxol, interferons, and platinum derivatives.

### Uses

Provided technologies, e.g., compounds, compositions, methods, etc., can provide various benefits through a number of mechanisms, including combinations thereof. Without the intention to be limited by any theory, in some embodiments, provided technologies achieve reduction in inflammation, e.g., inflammation in gastrointestinal tract, without general immune suppression, which a lot of existing immune system suppressor treatments for inflammatory bowl diseases do.

In some embodiments, the present disclosure provides methods for increasing level of a short-chain fatty acid in a system, comprising administering to the system a provided compound or a composition comprising a provided compound. In some embodiments, the present disclosure provides methods for delivering a short-chain fatty acid to a system, comprising administering to the system a provided compound or a composition comprising a provided compound.

In some embodiments, a system is a cell, tissue, organ, or subject. In some embodiments, a system is a cell. In some embodiments, a system is a tissue. In some embodiments, a system is an organ. In some embodiments, a system is a subject. In some embodiments, a system is a human.

In some embodiments, the present disclosure provides methods for increasing level of a short-chain fatty acid in a cell, tissue, and/or organ, comprising contacting a cell, tissue, and/or organ with a provided compound. In some embodiments, the present disclosure provides methods for increasing level of a short-chain fatty acid in a cell, comprising contacting a cell with a provided compound. In some embodiments, the present disclosure provides methods for increasing level of a short-chain fatty acid in a tissue, comprising contacting a tissue with a provided compound. In some embodiments, the present disclosure provides methods for increasing level of a short-chain fatty acid in an organ, comprising contacting an organ with a provided compound.

Without the intention to be limited by any theory, in some embodiments, after administration to a system, a provided compound is hydrolyzed and/or otherwise metabolized, in some embodiments partially or wholly enzymatically (e.g., through an esterase for compounds comprising ester groups), to provide one or more short-chain fatty acids.

In some embodiments, the present disclosure provides methods for preventing and/or treating a number of conditions, disorders or diseases that are associated with abnormal levels of one or more short-chain fatty acids, comprising administering to a subject susceptible thereto or suffering therefrom a provided compound or composition. In some embodiments, the present disclosure provides methods for preventing and/or treating a number of conditions, disorders or diseases that can benefit from increased levels of short-chain fatty acids, comprising administering to a subject susceptible thereto or suffering therefrom a provided compound or composition. As those skilled in the art appreciate, short-chain fatty acids can play a number of important roles associated with various conditions, disorders or diseases. For example, short-chain fatty acids participate in metabolism and energy production, can work as enzyme inhibitors (e.g., butyric acid as HDAC inhibitors), and/or may modulate functions of various receptors (e.g., G-coupled protein receptors, such as GPR41, GPR43 and GPR109a, etc.). Among other things, provided technologies can modulate these pathways, and/or inhibit functions of these proteins. In some embodiments, the present disclosure provides technologies for inhibiting an HDAC, comprising administering a provided compound or a composition thereof. In some embodiments, the present disclosure provides technologies for inhibiting an HDAC, comprising providing a provided compound or a composition thereof. In some embodiments, the present disclosure provides technologies for inhibiting an HDAC, comprising administering to a system comprising an HDAC a provided compound or a composition thereof. In some embodiments, the present disclosure provides methods for modulating functions of a G-coupled protein receptor, e.g., GPR41, GPR43 and GPR109a, etc., comprising providing a provided compound or a composition thereof. In some embodiments, the present disclosure provides methods for modulating functions of a G-coupled protein receptor, e.g., GPR41, GPR43 and GPR109a, etc., comprising administering to a system comprising a G-coupled protein receptor a provided compound or a composition thereof.

In some embodiments, a condition, disorder or disease is a gastrointestinal condition, disorder or disease.

In some embodiments, a condition, disorder or disease is an inflammatory bowel disease. In some embodiments, a condition, disorder or disease is ulcerative colitis. In some embodiments, a condition, disorder or disease is diversion colitis. In some embodiments, a condition, disorder or disease is radiation proctitis. In some embodiments, a condition, disorder or disease is radiation colitis. In some embodiments, a condition, disorder or disease is pouchitis. In some embodiments, a condition, disorder or disease is Crohn's disease. In some embodiments, a condition, disorder or disease is collagenous colitis. In some embodiments, a condition, disorder or disease is lymphocytic colitis.

In some embodiments, a condition, disorder or disease is an irritable bowel syndrome. In some embodiments, an irritable bowel syndrome is diarrhea type (IBS-D). In some embodiments, an irritable bowel syndrome is constipation type (IBS-C). In some embodiments, an irritable bowel syndrome is mixed type (IBS-M), wherein both diarrhea and constipation are common. In some embodiments, an irritable bowel syndrome is IBS-U, wherein neither diarrhea nor constipation is common.

In some embodiments, a subject suffers from or is susceptible to inadequate disease control and/or refractory disease. In some embodiments, a subject suffers from inadequate disease control. In some embodiments, a subject suffers from a refractory disease.

In some embodiments, provided compounds or compositions can be administered in combination with other therapies, for example, those for inflammatory bowel diseases and/or irritable bowel syndromes, etc. In some embodiments, a provided compound or composition is administered in combination with another therapy for an inflammatory bowel disease. In some embodiments, a provided compound or composition is administered in combination with another therapy for an irritable bowel syndrome. In some embodiments, when administered in combination with other therapies, provided compounds and/or compositions can be administered prior to, concurrently with, and/or subsequently to, other therapies. In some embodiments, when administered concurrently, a provided compound can be administered in the same composition, e.g., the same liquid, the same tablet, etc., as another therapy. Therapies for inflammatory bowel diseases and irritable bowel syndromes that can be used in combination with provided technologies are widely known and practiced in the art. For example, for inflammatory bowel diseases, exemplary therapies include anti-inflammatory drugs (e.g., corticosteroids and aminosalicylates, such as mesalamine (Asacol HD, Delzicol, etc.), balsalazide (Colazal) and olsalazine (Dipentum), etc.), immune system suppressors (which may work in a variety of ways to suppress the immune response that releases inflammation-inducing chemicals in the intestinal lining, and may work better as a combination of several; e.g., azathioprine (Azasan, Imuran), mercaptopurine (Purinethol, Purixan), cyclosporine (Gengraf, Neoral, Sandimmune), methotrexate (Trexall), infliximab (Remicade), adalimumab (Humira) golimumab (Simponi), natalizumab (Tysabri), vedolizumab (Entyvio), ustekinumab (Stelara), etc.), antibiotics (e.g., ciprofloxacin (Cipro) and metronidazole (Flagyl), etc.), surgery, etc. Exemplary therapies for irritable bowel syndromes include fiber supplements (e.g., psyllium (Metamucil), methylcellulose (Citrucel), etc.), anti-diarrheal medications (e.g., loperamide (Imodium), cholestyramine (Prevalite), colestipol (Colestid), colesevelam (Welchol), etc.), anticholinergic and/or antispasmodic medications (e.g., hyoscyamine (Levsin), dicyclomine (Bentyl), etc.), antibiotics, Alosetron (Lotronex), Lubiprostone (Amitiza), etc.

### EXAMPLES

Non-limiting examples are provided below. A person of ordinary skill in the art appreciates that other compounds, compositions and methods can similarly be prepared and performed in accordance with the present disclosure.

Various methods are widely known and practiced in the art, and can be utilized to prepare and/or test provided compound in accordance with the present disclosure. For example, a number of esterification methods can be used in accordance with the present disclosure as described in the examples in the present disclosure.

### Example 1

### Exemplary Procedure for Preparing Provided Compounds

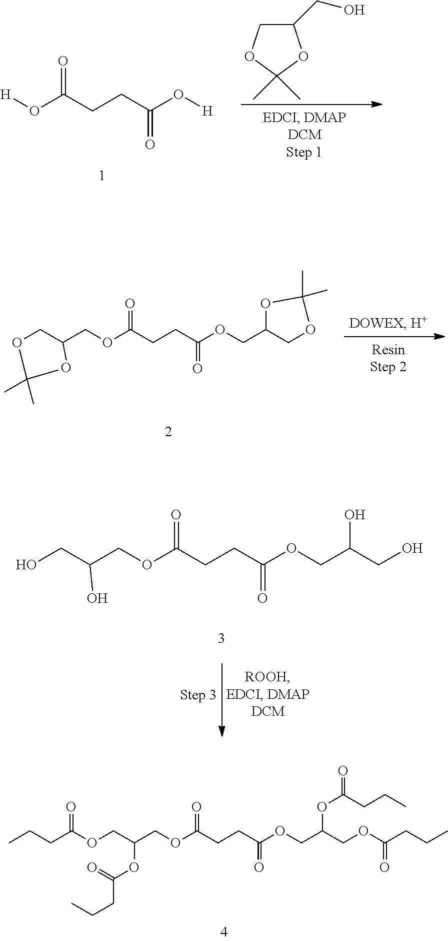

Experimental Procedure

Step 1: A suspension of succinic acid 1 (5 gm, 0.042 mol) in DCM (30 mL) was cooled to 0° C. To this was added solketal (11.74 gm, 0.089 mol) followed by addition of EDCI (25.89 gm, 0.14 mol) and DMAP (1.55 gm, 0.013 mol) at 0° C. The reaction was slowly warmed to room temperature and stirred overnight. The reaction mixture was diluted with ethyl acetate and washed with water (200 mL), sat. aq. sodium bicarbonate (200 mL) and brine (200 mL). The organic layer was dried over anhydrous sodium sulfate and concentrated. This crude residue was purified by column chromatography using hexanes and ethyl acetate (starting from 10% ethyl acetate and increased gradually to 40% ethyl acetate) to obtain 9.5 gm (65% yield) of colorless oil 2.

Step 2: To a cooled solution of compound 2 (9.5 gm) in methanol (130 mL) was added acidic resin (Amberlyst15 Hydrogen form, 20 gm). The reaction mixture was allowed to reach room temperature and stirred for 5 hrs. The resin was filtered off and the filtrate was concentrated and the concentrate was purified by column chromatography with increasing gradient of ethanol 1% to 20% in DCM to obtain 6 gm (82% Yield) of compound 3 as colorless syrupy liquid.

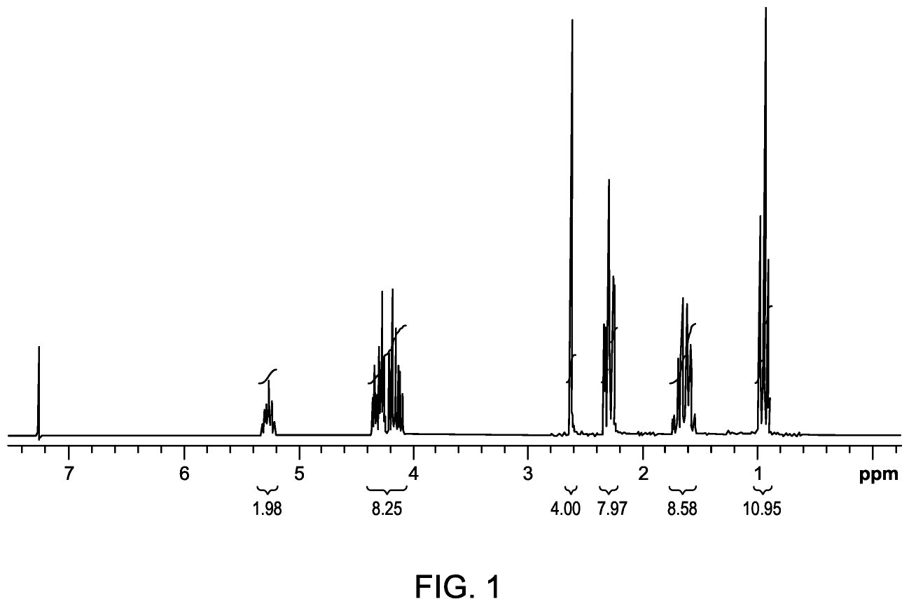

Step 3: To a solution of compound 3 (6 gm, 0.023 mol) in DCM (70 mL) was added butanoic acid (9.92 gm, 0.113 mol) at 0° C. followed by addition of EDCI (25.8 gm, 0.14 mol) and DMAP (1.65gm, 0.014 mol). The reaction mixture was allowed to reach room temperature and stirred overnight. The reaction mixture was diluted with ethyl acetate and washed with water (200 mL), sat. aq. sodium bicarbonate (200 mL) and brine (200 mL). The organic layer was dried over anhydrous sodium sulfate and concentrated. This crude residue was purified by column chromatography using hexanes and ethyl acetate (starting from 10% ethyl acetate and increased gradually to 40% ethyl acetate) to obtain 5.6 gm (45.5% Yield) of compound 4 (Compound I-1) as a liquid with >97% NMR purity. Example NMR spectra is presented in FIG. 1.

### Example 2

### Provided Compounds and Compositions Have Improved Properties

Properties of provided compounds and compositions can be readily tested, including using a number of methods widely known and practiced in the art, in accordance with the present disclosure. For example, viscosity and/or taste of provided compounds can be readily tested. In some embodiments, viscosity was assessed by testing whether a provided compound could readily flow out of a vial or be swirled. In some embodiments, viscosity was assessed by testing whether a provided compound could be administered by direct drinking by a subject. In some embodiments, viscosity can be measured quantitatively or qualitatively by methods and instruments as known and practiced in the art. In some embodiments, taste was assessed by testing whether a provided compound could be administered by direct drinking by a subject. In some embodiments, provided compounds, for example, I-1, have lower viscosity than glycerol, e.g., no more than 1200, 1100, 1000, 900, 800, 700, 600, 500, 400, 300, 200, 100, 90, 80, 70, 60, 50, 40, 30, 20, 10, 9, 8, 7, 6, 5, 4, 3, 2, or 1 cp, In some embodiments, provided compounds, have lower viscosity than glycerol, e.g., no more than 900, 800, 700, 600, 500, 400, 300, 200, 100, 90, 80, 70, 60, 50, 40, 30, 20, 10, 9, 8, 7, 6, 5, 4, 3, 2, or 1 cp at room temperature. In some embodiments, it was demonstrated that provided compounds are of greatly improved flow property and/or taste so that they can be much more easily formulated and administered compared to certain compounds with free carboxyl acid groups and/or hydroxyl groups (in some embodiments, for example, certain compounds with free carboxylic acid groups of succinic acid, were found to be very bitter and may not be as suitable for oral administration and/or patient compliance). In some embodiments, provided example compounds were demonstrated to be more palatable for oral formulations.

### Example 3

### Provided Compounds and Compositions are Effective in Disease Models

Among other things, provided technologies (compounds, compositions, methods, etc.) are particularly useful for treating conditions, disorders and diseases that are associated with reduced levels of short-chain fatty acids, and/or that can benefit from increased levels of short-chain fatty acids. As a person having ordinary skill in the art appreciates, many technologies can be utilized to assess activities and properties of provided compounds and compositions, and to demonstrates, among other things, usefulness, benefits and/or advantages, of provided compounds, compositions, and/or methods.

For example, as demonstrated herein, in mouse dextran sulfate sodium (DSS) model of acute ulcerative colitis, provided technologies delivered statistically significant benefits. An exemplary protocol is described below. As appreciated by those skilled in the art, various parameters may be adjusted according to known practices in the art.

Studies were conducted in accordance with The Guide for the Care & Use of Laboratory Animals (8^{th} Edition). A tested system was:

| | |
|---|---|
| Number of animals: | 45 study + 2 extra |
| Species/Strain or Breed: | Swiss Webster |
| Vendor: | Harlan |
| Age/Wt at Arrival: | 6-8 weeks old |
| Gender: | Female |
| Acclimation: | Acclimated for at least 4 days after arrival |
| Housing: | 5 animals/cage |

In a typical study, on study day −1, mice were weighed and randomized into treatment groups based on body weight. On study Day —O—, Groups 2-5 will be given 3% DSS in drinking water. Treatment was initiated and continue as indicated. Vehicle was water. On study day 5, DSS drinking water will be replaced with normal drinking water for the remainder of the study. On study day 7, animals will be anesthetized with Isoflurane and bled to exsanguination followed by cervical dislocation. The entire colon will be removed and measured. Further necropsy sample and data collection were performed as described.

An exemplary study group designation was described below.

| | | | | Dose Level | Dose | | Dosing | Dose Vol | Dose Conc |
|---|---|---|---|---|---|---|---|---|---|
| Group | N | Disease | Treatment | (mg/kg) | Route | Regimen^{1,2} | Days | (ml/kg)³ | (mg/ml) |
| 1 | 5 | N | Naive | N/A | N/A | N/A | N/A | N/A | N/A |
| 2 | 10 | Y | Vehicle | N/A | PO | BID | D0-D6 | 10 | N/A |
| 3 | 10 | Y | Cyclosporine | 75 | PO | QD | D0-D6 | 10 | 7.5 |
| | | | A (CsA) | | | | | | |
| 4 | 10 | Y | Compound I-1 | 2 g/kg | PO | BID | D0-D6 | 40 ul/mouse | 1 mg/μL⁴ |

¹BID dosing to occur at approximately 10-12 hr intervals
²QD dosing at approximately 24 hr intervals.
³The doses of test item to be administered were calculated daily in mg/kg based on the latest body weight of the animal PO.
⁴The drug is a 100% solution at a concentration of ~ 1 mg/μL

An exemplary study calendar was described below:

| Animal | | | | | | |
|---|---|---|---|---|---|---|
| Arrival | | | | | | |
| Day-1 | Day 0 | Day 1 | Day 2 | Day 3 | Day 4 | Day 5 |
| weigh, | weigh, | weigh, dose | weigh, dose | weigh, dose | weigh,dose | weigh, dose, |
| randomize | begin dss, | | | | | replace DSS with |
| | dose | | | | | normal drinking water |
| Day 6 | Day 7 | | | | | |
| weigh, dose | weigh, | | | | | |
| | necropsy | | | | | |

For necropsy, sacrifice schedule was Day 7. Method of Euthanasia was CO₂ asphyxiation followed by cervical dislocation. Colon length, weight, and score data were collected. An exemplary scoring system: at necropsy, assess for clinical evidence of blood or blood-tinged fluid for evaluation of colon content scores. Colon content scored at necropsy according to the following criteria: 0=normal, no blood observed; 1=semi-solid stool, may be slightly blood tinged; 2=semi-solid to fluid stool with definite evidence of blood; 3=bloody fluid or no content, (include animals with no observable distal content in this category). For necropsy tissue sample collection: Type: colon; Gr/An: all; Details: proximal and distal halves; Storage Condition: 10% NBF; Disposition: Histology.

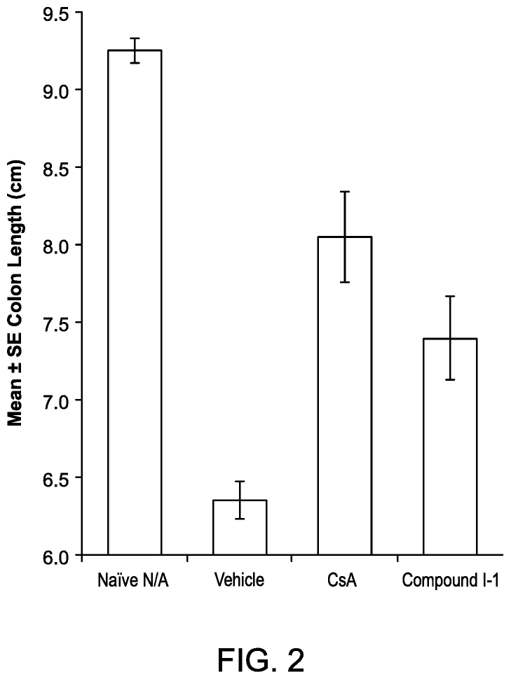

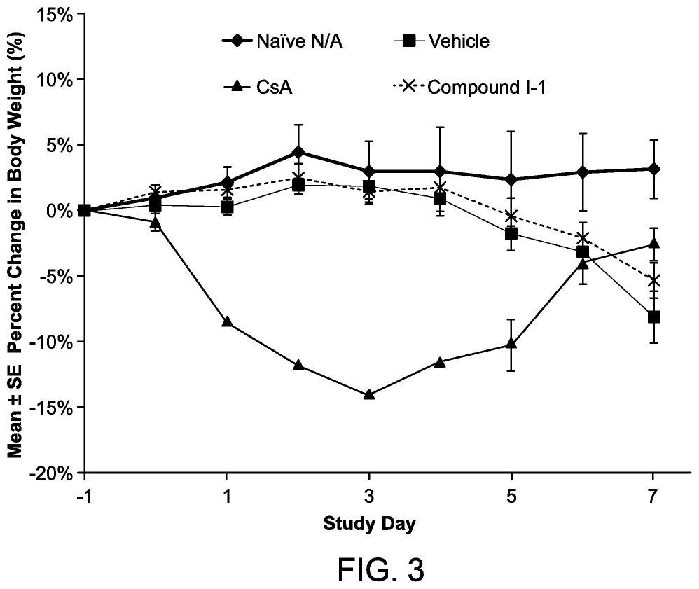

Example data from a study were presented: colon data were described in Tables 1 and 2, and FIG. 2, and body weight data in FIG. 3.

TABLE 1
Collected colon data from a study.

| | | | | Colon |
|---|---|---|---|---|
| | Colon | | | Content |
| | Length | Wet Wt | Weight:Length | Score |
| | (cm) | (g) | (g/cm) | (0-3) |
| Group 1 | | | | |
| Naïve | | | | |
| N/A | | | | |
| 1 | 9.25 | 0.243 | 0.026 | 0 |
| 2 | 9.50 | 0.261 | 0.027 | 0 |
| 3 | 9.25 | 0.269 | 0.029 | 0 |
| 4 | 9.25 | 0.237 | 0.026 | 0 |
| 5 | 9.00 | 0.255 | 0.028 | 0 |
| Group 2 | | | | |
| Vehicle | | | | |
| PO, BID (d 0-6) | | | | |
| 1 | 6.25 | 0.335 | 0.054 | 2 |
| 2 | 6.50 | 0.261 | 0.040 | 1 |
| 3 | 6.75 | 0.350 | 0.052 | 1 |
| 4 | 7.00 | 0.428 | 0.061 | 2 |
| 5 | 6.25 | 0.308 | 0.049 | 2 |
| 6 | 5.75 | 0.291 | 0.051 | 1 |
| 7 | 6.00 | 0.294 | 0.049 | 1 |
| 8 | 6.50 | 0.349 | 0.054 | 2 |
| 9 | 6.00 | 0.304 | 0.051 | 1 |
| 10 | 6.50 | 0.371 | 0.057 | 2 |
| Group 3 | | | | |
| CsA (75 mg/kg) | | | | |
| PO, QD (d 0-6) | | | | |
| 1 | 6.50 | 0.246 | 0.038 | 1 |
| 2 | 8.00 | 0.316 | 0.040 | 1 |
| 3 | 9.00 | 0.304 | 0.034 | 1 |
| 4 | 8.25 | 0.288 | 0.035 | 1 |
| 5 | 8.75 | 0.246 | 0.028 | 1 |
| 6 | 8.50 | 0.236 | 0.028 | 0 |
| 7 | 7.50 | 0.262 | 0.035 | 1 |
| 8 | 8.75 | 0.227 | 0.026 | 1 |
| 9 | 6.50 | 0.302 | 0.046 | 1 |
| 10 | 8.75 | 0.283 | 0.032 | 1 |
| Group 4 | | | | |
| Compound I-1 | | | | |
| (2 g/kg) | | | | |
| PO, BID (d0-6) | | | | |
| 1 | 8.00 | 0.369 | 0.046 | 1 |
| 2 | 8.25 | 0.320 | 0.039 | 1 |
| 3 | 8.00 | 0.343 | 0.043 | 1 |
| 4 | 6.25 | 0.391 | 0.063 | 1 |
| 5 | 8.00 | 0.332 | 0.042 | 1 |
| 6 | 6.25 | 0.339 | 0.054 | 2 |
| 7 | 8.50 | 0.291 | 0.034 | 1 |
| 8 | 7.00 | 0.332 | 0.047 | 1 |
| 9 | 7.00 | 0.390 | 0.056 | 2 |
| 10 | 6.75 | 0.324 | 0.048 | 1 |

TABLE 2
Certain information of FIG. 2.

| Tukey's multiple | Mean | 95.00% | | Adjusted |
|---|---|---|---|---|
| comparisons test | Diff. | CI of diff. | Significant? | P Value |
| Naïve vs. Vehicle | 2.9 | 1.848 to 3.952 | Yes | <0.0001 |
| Naïve vs. CsA | 1.2 | 0.148 to 2.252 | Yes | 0.0205 |
| Naïve vs. | 1.85 | 0.798 to 2.902 | Yes | 0.0002 |
| Compound I-1 | | | | |
| Vehicle vs. CsA | −1.7 | −2.559 to −0.841 | Yes | <0.0001 |
| Vehicle vs. | −1.05 | −1.909 to −0.191 | Yes | 0.0118 |
| Compound I-1 | | | | |
| CsA vs. | 0.65 | −0.209 to 1.509 | No | 0.1908 |
| Compound I-1 | | | | |

As illustrated by data presented herein, e.g., Table 1, Table 2 and FIG. 2, etc., provided compounds such as compound I-1 effectively restored colon length and demonstrated efficacy. As shown herein, provided compounds can be successfully administered orally, and can be administered at sufficiently high dosage to provide desired benefits. Compared to compounds like cyclosporine which is an immunomodulator, provided compounds can provide significant benefits while having much less or no impact on the immune system.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

### Example 4

Compounds of the invention were tested for the ability to treat 2,4,6-trinitrobenzene sulphonic acid (TNBS)-induced colitis. Mice with TNBS-induced colitis were given CV-8784 (also referred to as CTP-06) orally twice daily. CV-8784 has the following structure:

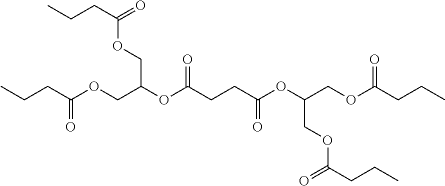 [SMILES: CCCC(=O)OCC(COC(=O)CCC)OC(=O)CCC(=O)OC(COC(=O)CCC)COC(=O)CCC]

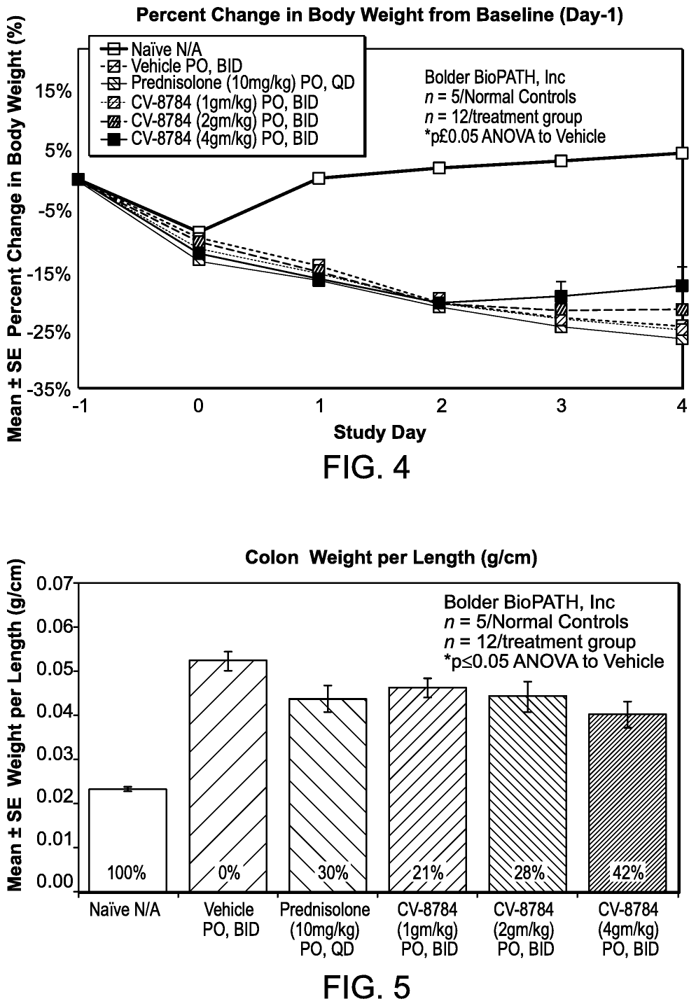

FIG. 4 is a graph of change in body weight over time in animals with TNBS-induced colitis after administration of CV-8784 or control compounds. White squares represent naive animals. Remaining squares represent animals with TNBS-induced colitis treated as follows: grey squares, vehicle alone twice per day; green squares, 10 mg/kg prednisolone once per day; dark blue squares, 1 g/kg CV-8784 twice per day; light blue squares, 2 g/kg CV-8784 twice per day; and yellow squares, 4 g/kg CV-8784 twice per day.

FIG. 5 is a graph of colon weight per length in animals with TNBS-induced colitis after administration of CV-8784 or control compounds. White bar represents naive animals. Remaining bars represent animals with TNBS-induced colitis treated as follows: grey bar, vehicle alone twice per day; green bar, 10 mg/kg prednisolone once per day; dark blue bar, 1 g/kg CV-8784 twice per day; light blue bar with cross-hatching, 2 g/kg CV-8784 twice per day; and yellow bar with cross-hatching, 4 g/kg CV-8784 twice per day.

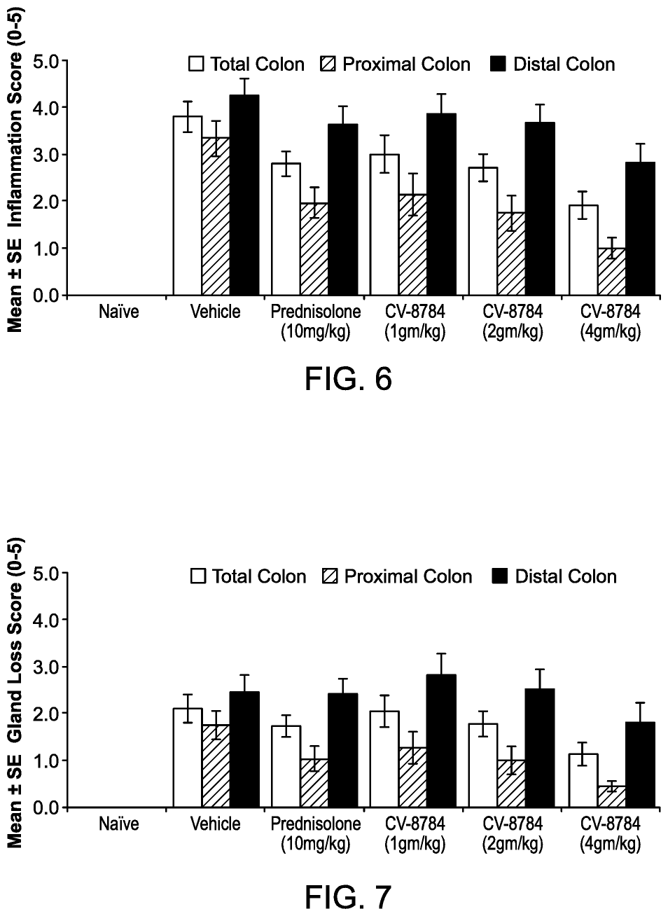

FIG. 6 is a graph of the overall inflammation score in colon tissue in animals with TNBS-induced colitis after administration of CV-8784 or control compounds. White bars represent inflammation in total colon; grey bars represent inflammation in proximal colon, and black bars represent inflammation in distal colon. Animal populations are same as described in relation to FIGS. 4 and 5 and are indicated below X-axis.

FIG. 7 is a graph of gland loss in colon tissue in animals with TNBS-induced colitis after administration of CV-8784 or control compounds. White bars represent gland loss in total colon; grey bars represent gland loss in proximal colon, and black bars represent gland loss in distal colon. Animal populations are same as described in relation to FIGS. 4 and 5 and are indicated below X-axis.

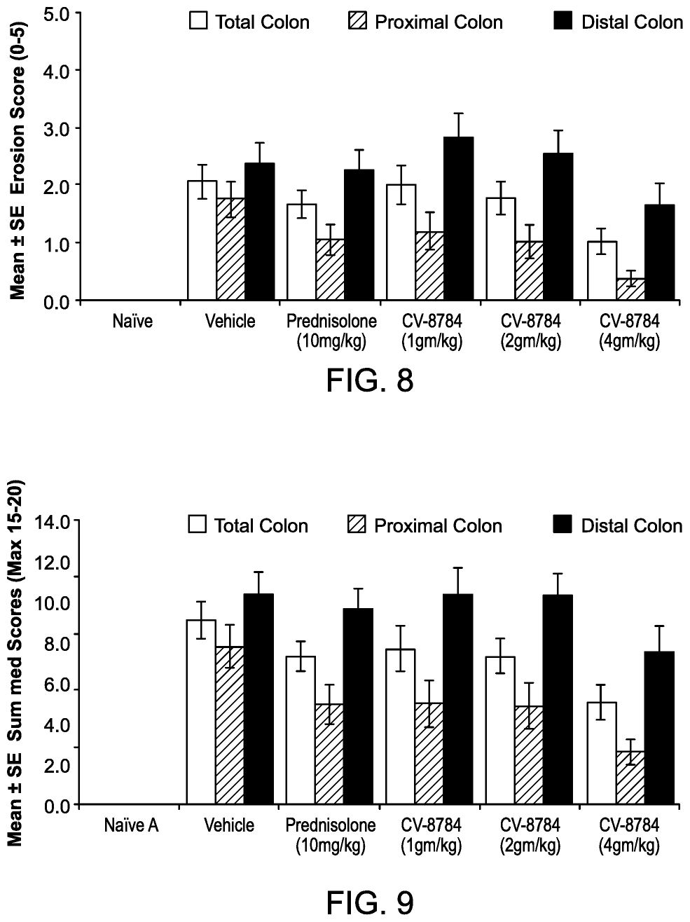

FIG. 8 is a graph of tissue erosion in colon tissue in animals with TNBS-induced colitis after administration of CV-8784 or control compounds. White bars represent erosion in total colon; grey bars represent erosion in proximal colon, and black bars represent erosion in distal colon. Animal populations are same as described in relation to FIGS. 4 and 5 and are indicated below X-axis.

FIG. 9 is a graph of edema in colon tissue in animals with TNBS-induced colitis after administration of CV-8784 or control compounds. White bars represent edema in total colon; grey bars represent edema in proximal colon, and black bars represent edema in distal colon. Animal populations are same as described in relation to FIGS. 4 and 5 and are indicated below X-axis.

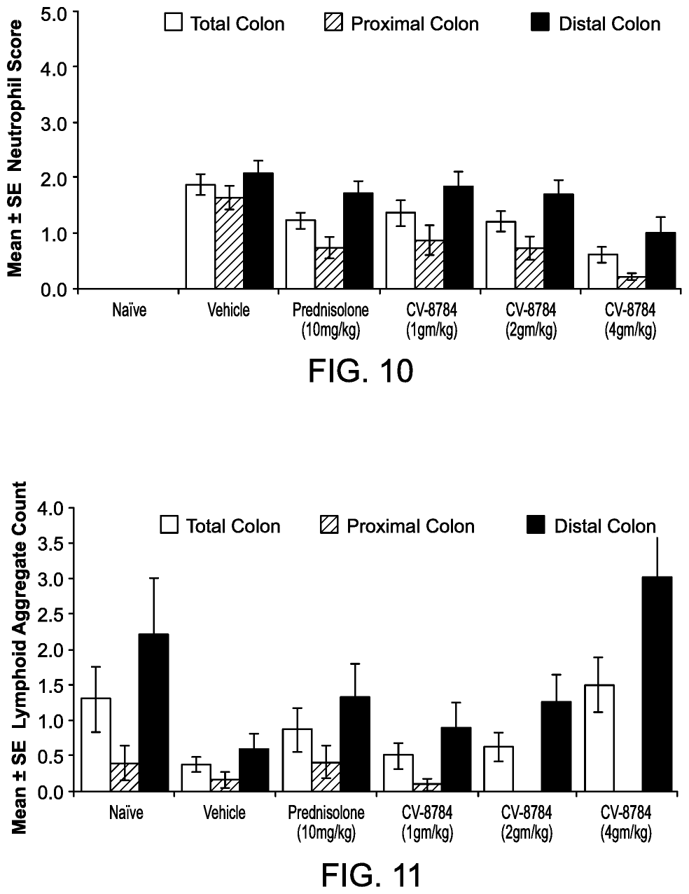

FIG. 10 is a graph of neutrophil infiltration in colon tissue in animals with TNBS-induced colitis after administration of CV-8784 or control compounds. White bars represent neutrophil infiltration in total colon; grey bars represent neutrophil infiltration in proximal colon, and black bars represent neutrophil infiltration in distal colon. Animal populations are same as described in relation to FIGS. 4 and 5 and are indicated below X-axis.

FIG. 11 is a graph of lymhocyte aggregation in colon tissue in animals with TNBS-induced colitis after administration of CV-8784 or control compounds. White bars represent lymhocyte aggregation in total colon; grey bars represent lymhocyte aggregation in proximal colon, and black bars represent lymhocyte aggregation in distal colon. Animal populations are same as described in relation to FIGS. 4 and 5 and are indicated below X-axis.

### Example 5

Compounds of the invention were tested for the ability to treat dextran sodium sulfate (DSS)-induced colitis. Mice with DSS-induced colitis were given CTP-06 (also called CV-8784) orally twice daily.

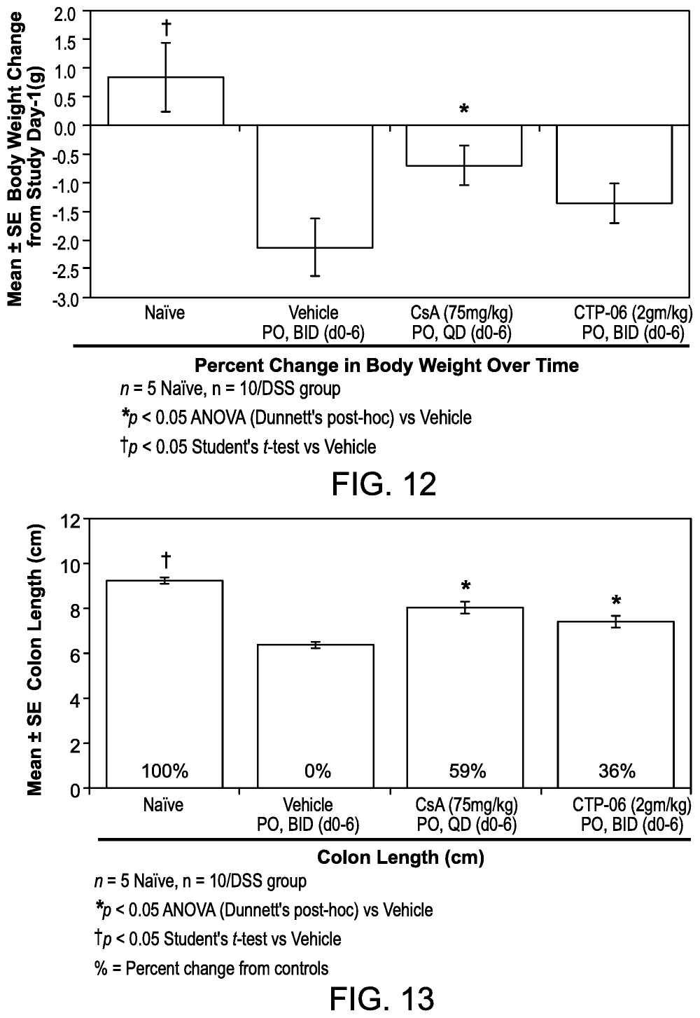

FIG. 12 is a graph of change in body weight over time in animals with DSS-induced colitis after administration of CTP-06 or control compounds. Animals were either naive or given orally vehicle alone twice per day, 75 mg/kg cyclosporin A once per day, or 2 mg/kg CTP-06 twice per day, as indicated below X-axis.

FIG. 13 is a graph of colon length in animals with DSS-induced colitis after administration of CTP-06 or control compounds. Animals were either naive or given orally vehicle alone twice per day, 75 mg/kg cyclosporin A once per day, or 2 mg/kg CTP-06 twice per day, as indicated below X-axis.

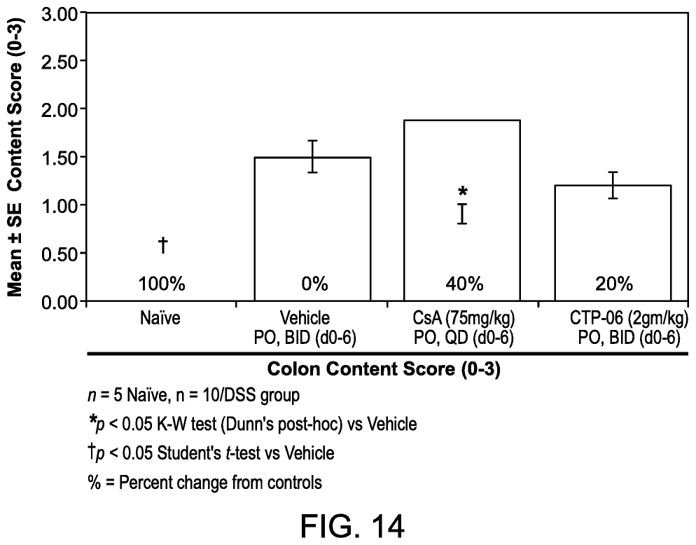

FIG. 14 is a graph of colon content in animals with DSS-induced colitis after administration of CTP-06 or control compounds. Animals were either naive or given orally vehicle alone twice per day, 75 mg/kg cyclosporin A once per day, or 2 mg/kg CTP-06 twice per day, as indicated below X-axis.

### Example 6

Compounds of the invention were tested for the ability to treat morphine-induced suppression of coonic propulsion. Mice were given CV-8784 (also called CTP-06) or control substances by oral gavage. Thirty minutes after oral gavage, 2 mg/kg morphine was administered subcutaneously. Thirty minutes after morphine administration, a 3 mm glass bead was administered, and time to evacuation of the bead was monitored.

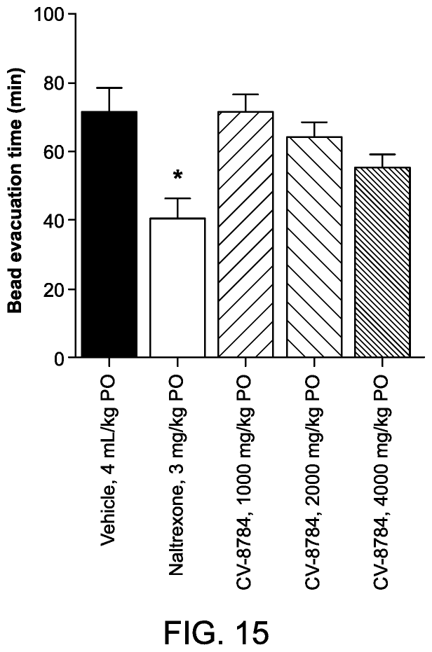

FIG. 15 is a graph of intestinal transit of a glass bead in animals after administration of CV-8784 or control compounds. Animals were given following treatments orally, as indicated below X-axis: black bar, 4 ml/kg vehicle alone; white bar, 3 mg/kg naltrexone; left-downward cross-hatched bar, 1000 mg/kg CV-8784; right-downward cross-hatched bar, 2000 mg/kg CV-8784; and crisscross-hatched bar, 4000 mg/kg CV-8784. Cohorts treated with vehicle or naltrexone included 10 animals, and cohorts treated with CV-8784 included 15 animals.

### Example 7

Compounds of the invention were tested for their effects on intestinal mobility. Mice were given CV-8784 (also called CTP-06) or control substances by oral gavage. Sixty minutes after oral gavage, 0.3 ml of 5% charcoal/10% gum arabic solution was given orally. Animals were sacrificed 15 minutes after administration of the 5% charcoal/10% gum arabic solution, and the distance of charcoal migration from the pyloris into the intestine was measured.

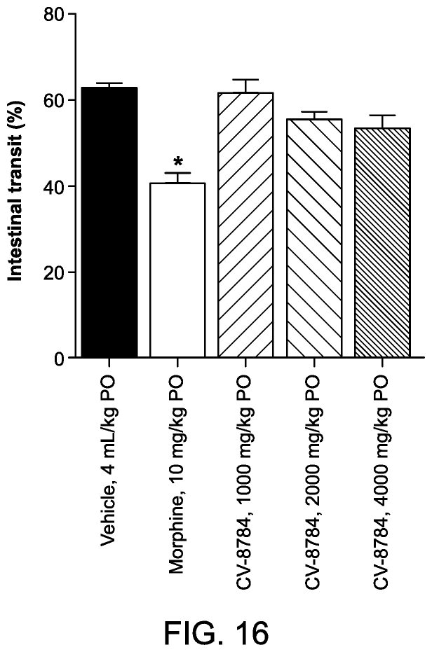

FIG. 16 is a graph of intestinal transit of a charcoal solution in an animals after administration of CV-8784 or control compounds. Animals were given following treatments orally, as indicated below X-axis: black bar, 4 ml/kg vehicle alone; white bar, 10 mg/kg moprhine; left-downward cross-hatched bar, 1000 mg/kg CV-8784; right-downward cross-hatched bar, 2000 mg/kg CV-8784; and crisscross-hatched bar, 4000 mg/kg CV-8784. Cohorts treated with vehicle or naltrexone included 5 animals, and cohorts treated with CV-8784 included 10 animals.

1. A compound having the structure of formula I:
R^{A}-L-R^{B}, I
or a salt thereof, wherein:
R^{A} is 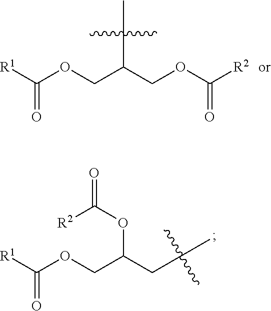
R^{B} is 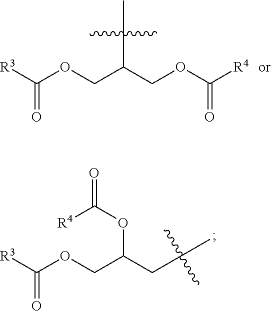
each of R¹, R², R³ and R⁴ is independently C₁₋₃ alkyl;
L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with bivalent C₁₋₄ aliphatic, —O—, —C(O)—, —C(O)O—, —S—, —N(R′)—, or —C(O)N(R′)—;
R′ is R or —C(O)R;
each R is independently hydrogen, or an optionally substituted group selected from C₁₋₆ aliphatic, C₁₋₆ heteroaliphatic having 1-5 hetereoatoms independently selected from nitrogen, oxygen, and sulfur, phenyl, 5-6 membered heteroaryl having 1-5 hetereoatoms independently selected from nitrogen, oxygen, and sulfur, and 3-6 membered heterocyclyl having 1-5 hetereoatoms independently selected from nitrogen, oxygen, and sulfur.

2. The compound of claim 1, wherein R^{A} is

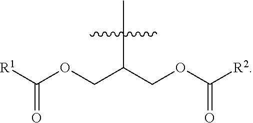

3. The compound of claim 1, wherein R^{A} is

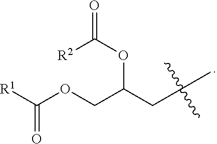

4. The compound of claim 1, wherein R^{B} is

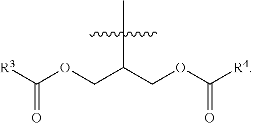

5. The compound of claim 1, wherein R^{B} is

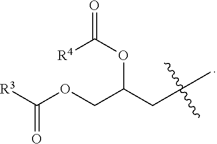

6. The compound of claim 1, wherein R¹ is methyl.

7. The compound of claim 1, wherein R¹ is ethyl.

8. The compound of claim 1, wherein R¹ is n-propyl.

9. The compound of claim 1, wherein R² is methyl.

10. The compound of claim 1, wherein R² is ethyl.

11. The compound of claim 1, wherein R² is n-propyl.

12. The compound of claim 1, wherein R³ is methyl.

13. The compound of claim 1, wherein R³ is ethyl.

14. The compound of claim 1, wherein R³ is n-propyl.

15. The compound of claim 1, wherein R⁴ is methyl.

16. The compound of claim 1, wherein R⁴ is ethyl.

17. The compound of claim 1, wherein R⁴ is n-propyl.

18. The compound of claim 1, wherein L is an optionally substituted, linear or branched, bivalent group selected from C₁₋₆ aliphatic and C₁₋₆ heteroaliphatic having 1-5 hetereoatoms independently selected from nitrogen, oxygen, and sulfur, wherein one or more methylene units of the aliphatic and heteroaliphatic are optionally and independently replaced with bivalent C₁₋₄ aliphatic, —O—, —C(O)—, or —C(O)O—.

19. The compound of claim 1, wherein L is an optionally substituted, linear or branched, bivalent C₁₋₆ aliphatic, wherein one or more methylene units of the aliphatic and heteroaliphatic are optionally and independently replaced with bivalent C₁₋₄ aliphatic, —O—, —C(O)—, or —C(O)O—.

20. The compound of claim 1, wherein L is an optionally substituted, linear or branched, bivalent C₁₋₆ aliphatic, wherein one or more methylene units of the aliphatic and heteroaliphatic are optionally and independently replaced with bivalent —O—, —C(O)—, or —C(O)O—.

21-69. (canceled)
